(19) United States
(12) Patent Application Publication
(10) Pub. No.: US 2009/0156441 A1
(43) Pub. Date: Jun. 18, 2009

(54) Cycloalkyl phenylenediamines as deposit control agents for lubricants

(76) Inventors: Robert G. Rowland, Woodbridge, CT (US); Theodore E. Nalesnik, Hopewell Junction, NY (US); Joseph P. Hartley, Cumnor (GB); Neal J. Milne, Ramsbury (GB); Paul Wilson, Abingdon (GB)

Correspondence Address: Daniel Reitenbach;CHEMTURA CORPORATION, 199 Benson Road, Middlebury, CT, 06749, US

(21) Appl. No.: 12/001,951

(22) Filed: Dec. 12, 2007

(51) Int. Cl. C10M 135/36; C07D 211/06; C07C 211/49; C09K 5/00; C10L 1/22; C10L 1/223; C10L 1/232; C10M 133/12; C10M 133/40

(52) U.S. Cl. 508/251; 546/229; 564/431; 252/68; 508/563; 508/262

(57) ABSTRACT

The invention is a C₅-C₁₂ cycloalkyl substituted phenylenediamine that provides deposit-control lubricant additives for organic materials including lubricating oil, gasoline, and diesel fuels. The invention improves the oxidative stability of the lubricants and fuels by adding cycloalkyl substituted phenylenediamines having one or more additional N-substituents.

### BACKGROUND OF THE INVENTION

1. Field of the Invention

The present invention relates to reducing the tendency of lubricants, especially fully-formulated hydrocarbon based lubricating oils, to form engine deposits, and improving the oxidative stability of the lubricants. Specifically, cycloalkyl substituted phenylenediamines with one or more additional N-substituents are added to the lubricants.

2. Description of Related Art

Lubricants, such as those used in a variety of machinery, are susceptible to oxidative deterioration during storage, transportation, and usage, particularly when such lubricants are exposed to high temperatures and iron catalytic environments, which greatly promote their oxidation. This oxidation, if not controlled, contributes to the formation of corrosive acidic products, sludge, varnishes, resins, and other oil-insoluble products and may lead to a loss of designated physical and tribological properties of the lubricants. These oxidation products may lead to the formation of harmful deposits on critical engine parts, such as the pistons, piston liners, valves, and valve lifters. It is therefore a common practice to include deposit-control and antioxidant additives in lubricants to prevent, at least to some extent, oxidation, so as to extend the useful life of the lubricants.

Lubricant compositions containing various secondary diarylamines as antioxidants are widely known in the art. The use of para-phenylenediamines is also known. Para-phenylenediamines have more commonly,been employed as motor fuel stabilizers and antiozonants and antioxidants for rubber.

U.S. Pat. No. 2,451,642 discloses ortho-, meta-, and para-phenylenediamine as useful antioxidants for lubricating oil compositions for use in environments where iron-catalyzed oxidation reaction can take place. N,N′-dimethyl-ortho-phenylenediamine, N,N′-dimethyl-meta-phenylenediamine, lauryl-meta-phenylenediamine, N,N′-dicyclohexyl-para-phenylenediamine, and various di-and tetra-n-alkyl-para-phenylenediamines are similarly disclosed.

U.S. Pat. No. 2,718,501 discloses a stabilizer system consisting of an aromatic amine with at least two aromatic rings, including N,N′-diphenyl-para-phenylenediamine, and an organic aliphatic sulfur compound, which is said to be suitable for stabilizing mineral hydrocarbon lubricating oils, synthetic hydrocarbon oils, and polyalkylene glycol oils.

U.S. Pat. No. 2,857,424 discloses the preparation of oxalic acid salts of fuel stabilizing N,N′-dialkyl-para-phenylenediamines as a way of rendering the additives less toxic. The preparation of the oxalate salt of N,N′-dicyclohexyl-para-phenylenediamine is disclosed. The preparation of the oxalate salts of other unspecified dicycloalkyl ortho-, meta-, and para-phenylenediamines is contemplated.

U.S. Pat. No. 2,883,362 discloses the stabilization of rubber towards cracking by the addition of N,N,N′,N′-tetraalkyl para-phenylenediamines. The only such compound disclosed wherein one or more of the alkyl groups is cycloalkyl is N,N′-dicyclohexyl-N,N′-dimethyl-para-phenylenediamine.

Great Britain Patent No. 835,826 discloses the reaction of certain phenylenediamines with alkyldihalides to make higher molecular weight compounds that are useful as antiozonants for rubber. N,N′-dicyclohexyl-ortho-phenylenediamine, N,N′-dicyclohexyl-para-phenylenediamine, N,N′-dicyclohexyl-N-methyl-ortho-phenylenediamine, and N,N′-dicyclohexyl-N-methyl-para-phenylenediamine are disclosed as being suitable starting materials for this reaction.

U.S. Pat. No. 3,211,793 claims the preparation of N,N′-dicyclohexyl-N-isobutenyl-para-phenylenediamine, exemplifying the utility as an antioxidant for rubber. The patent also suggests that the isobutenyl-para-phenylene diamines of the invention would be useful as antioxidants for fuels and oils. This patent relates to the preparation of N,N,N′-tricycloalkyl-N-isobutenyl-para-phenylenediamines. However, this patent fails to recognize the exceptional anti-deposit activity that cycloalkyl groups, and particularly cyclohexyl groups, impart to N-alkyl-phenylenediamine additives. The patent further fails to anticipate the use of isobutenyl-para-phenylene diamines in fully formulated lubricants and fully additized fuels.

U.S. Pat. No. 3,304,285 discloses a synergistic mixture of N,N′-dicycloalkyl-para-phenylenediamine with N-phenyl-N-alkylphenyl-para-phenylenediamines for use as a stabilizer for vulcanizable diene rubber. The patent relates to the use of symmetric dicycloalkyl para-phenylene diamines from dicyclopropyl-para-phenylenediamine through dicyclodecyl-para-phenylenediamine but only exemplifies the dicyclopentyl- and dicyclohexyl-compounds.

Oberster, A. E. et al., 45 CAN. J. CHEM. 195-201 (1967), prepared 39 novel phenylenediamines as part of a program to find antiozonants for rubber that are not sensitizers or dermatotoxic. In some compounds the N′-phenylenediamine nitrogen was variously fused into a pyrrolidine, piperidine, hexamethyleneimine (homopiperidine), morpholine, or 2,6-dimethylmorpholine ring. In each case the N-cyclohexyl compound was prepared.

U.S. Pat. No. 3,402,201 discloses N,N′-dicyclooctyl-para-phenylenediamine as a stabilizer for organic materials, particularly rubber, and exemplifies its use as a gasoline inhibitor. The similar use of N,N′-dicyclooctyl-ortho- and -meta-phenylenediamines is contemplated.

Great Britain Patent No. 1,296,592 discloses N-aryl, N-alkyl-N′-alkyl-N′-cycloalkyl-para-phenylenediamines, where aryl is phenyl or alkylphenyl, alkyl is an alkyl group containing from one to four carbons, and the cycloalkyl group contains from five to nine carbons. These compounds are useful as antioxidants for peroxide-crosslinked polyethylene.

Makogon, A. N. et al., 12 KHEMICHESKAYA PROMYSHLENNOST, SERIYA: METODY ANALIZA I KONTROLYA KACHESTVA PRODUKTSII V KHIMICHESKOI PROMYSHLENNOSTI 18-21 (1980), describe the characterization of N,N′-dialkyl-para-phenylenediamines reaction products obtained by the catalytic alkylation of 4-aminodiphenylamine with C₇-C₉ alcohols.

U.S. Pat. No. 4,487,759 discloses the use of certain N,N′,N′-trialkyl-N-phenyl-para-phenylenediamines (e.g., N,N′-didecyl-N′-octyl-N-phenyl-para-phenylenediamine) as light stabilizers for unsaturated insect pheromones that are contained in a micro-encapsulation delivery system.

U.S. Pat. Nos. 5,207,939 and 5,312,461 disclose certain Mannich base reaction products of mono- or dialkyl-phenylenediamines, an aldehyde or ketone, and a hindered phenol, which can be used in an antioxidant amount in lubricating oils, greases, and fuel compositions.

Japan Patent No. 59-020,392 discloses a lubricant composition comprising N,N′-di-sec-butyl-para-phenylenediamine for pressure forming of oil tanks. The lubricant composition also contains hindered phenolic antioxidant.

U.S. Pat. No. 5,711,767 discloses the use of certain phenylenediamines in combination with nitroxides as stabilizers for gasoline. The following ortho-phenylenediamines are claimed: N,N′-di-sec-butyl-ortho-phenylenediamine, N,N′-di-(1,4-dimethylpentyl)-ortho-phenylenediamine, and N-sec-butyl-N′-phenyl-ortho-phenylenediamine. The following cyclohexyl phenylenediamines are claimed: N-cyclohexyl-N′-phenyl-para-phenylenediamine, N,N′-dicyclohexyl-para-phenylenediamine. The detailed disclosure states that the 1,4-dimethylpentyl compounds are the more preferred of these phenylenediamines for this application.

U.S. Publication No. 2006/0128574 discloses the use of secondary diarylamines in combination with N,N′-dialkyl-para-phenylenediamines, and optionally hindered phenolics, as stabilizers for lubricants and fuels. The following cyclohexyl phenylenediamines are claimed: N-cyclohexyl-N′-phenyl-para-phenylenediamine, N,N′-dicyclohexyl-para-phenylenediamine.

U.S. Publication No. 2006/0189824. A1 discloses various N-alkyl-N-(dialkylhydroxyphenyl)alkyl-N′-phenyl-para-phenylene diamines, methods for their preparation by Mannich reactions of dialkylphenols with N-phenyl-para-phenylenediamines, and their use as antioxidants.

U.S. Publication No. 2007/0006855 A1 discloses the use of alkylated para-phenylenediamines as soot dispersants in passenger car and heavy-duty diesel engines equipped with exhaust gas recirculation systems (EGR). The patent anticipates an extremely broad scope of polyalkylated para-phenylenediamines for this specific application alone.

The foregoing disclosures are incorporated by reference.

While phenylenediamines are known to act effectively as antioxidants, these compounds have been found to be disadvantageous commercially, since the presence of such compounds, when used in amounts conventionally used to provide antioxidancy, displayed adverse effects on piston deposit and varnish control and also displayed aggressiveness toward fluoroelastomeric engine seal materials. These adverse effects are particularly apparent with phenylenediamine compounds having higher nitrogen contents (compounds having relatively small hydrocarbyl substituents). Recent lubricating oil specifications for PCDO set by original equipment manufacturers (OEMs) have required reduced levels of lubricant phosphorus (e.g., less than 800 ppm). To date, lubricating oil specifications for heavy duty diesel (HDD) engines have not limited phosphorus content, although the next generation of lubricant specifications (e.g., API CJ-4) is expected to do so. Expected limits on phosphorus content (such as to 1200 ppm or less), and reductions in the allowable amounts of sulfated ash (SASH) and sulfur will limit the amount of zinc dialkyldithiophosphate (ZDDP), one of the most cost-effective antiwear/antioxidant compounds, that a lubricant formulator can use.

### SUMMARY OF THE INVENTION

It is an object of this invention to reduce the tendency of lubricants to form engine deposits by adding thereto cycloalkyl substituted phenylenediamines with one or more additional N-substituents.

It is a further object of this invention to improve the oxidative stability of said lubricants, by adding thereto cycloalkyl substituted phenylenediamines with one or more additional N-substituents.

The present invention thus comprises a composition for a lubricating oil, grease, fuel, or functional fluid subject to oxidative degradation wherein the composition comprises an amount of an N-cycloalkyl substituted phenylenediamine additive in a quantity sufficient to stabilize the fluid against the formation of deposits.

Specifically, the present invention is directed to C₅-C₁₂ cycloalkyl substituted phenylenediamines, which are substantially better deposit-control lubricant additives for gasoline and diesel engines than even closely related acyclic phenylenediamines. The improved deposit control can be measured in the Mid-High Temperature Thermo-oxidation Engine Oil Simulation Test (TEOST) MHT.

More particularly, the present invention is directed to a compound, a method for stabilizing a composition, and the composition comprising a fluid subject to oxidative degradation and an amount of at least one N-cycloalkyl-substituted phenylenediamine additive sufficient to stabilize said fluid against the formation of deposits, wherein said additive is of the structure:

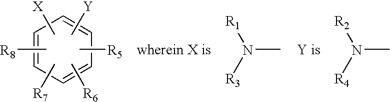

R₁ is a cycloalkyl ring selected from the group consisting of:

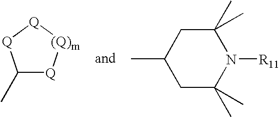

wherein

R₅, R₆, R₇, and R₈ are independently selected from the group consisting of hydrogen, linear and branched alkyl groups of from 1 to 20 carbon atoms, cycloalkyl, allyl, methyallyl, isobutenyl, alkenyl, furfuryl, benzyl, alkaryl, and phenyl;

each Q is C(R₉)(R₁₀), wherein each R₉ and R₁₀ is independently selected from the group consisting of hydrogen, linear and branched alkyl groups of from 1 to 20 carbon atoms, and cycloalkyl;

wherein R₁₁ is H, linear and branched alkyl groups of from 1 to 20 carbon atoms, cycloalkyl, aryl, alkaryl, and O; and wherein Q and m are as defined above;

and

m is an integer of 1 to 8; and

wherein, when X and Y are para to each other, R₂, R₃, and R₄ are in an interrelated relationship selected from the group consisting of:

(A) R₂ is a cycloalkyl ring independently selected from the group consisting of:

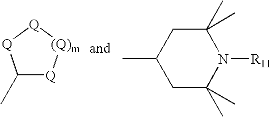

wherein

each Q is C(R₉)(R₁₀), wherein each R₉ and R₁₀ is independently selected from the group consisting of hydrogen, linear and branched alkyl groups of from 1 to 20 carbon atoms, and cycloalkyl;

wherein R₁₁ is H, linear and branched alkyl groups of from 1 to 20 carbon atoms, cycloalkyl, aryl, alkaryl, and O; and wherein Q and m are as defined above;

R₃ is an independently selected cycloalkyl ring of the structure:

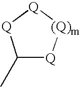

wherein Q and m are as defined above;
and

R₄ is selected from the group consisting of linear or branched alkyl of from 1 to about 36 carbon atoms, aryl, alkaryl, cycloalkyl, and alkenyl of from 3 to about 36 carbon atoms, all optionally containing a nitrogen, oxygen, or sulfur atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not —CH₂CH₂— or —CH₂CH(CH₃)—;

(B) R₂ is a cycloalkyl ring independently selected from the group consisting of:

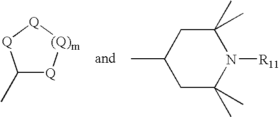

wherein

each Q is C(R₉)(R₁₀), wherein each R₉ and R₁₀ is independently selected from the group consisting of hydrogen, linear and branched alkyl groups of from 1 to 20 carbon atoms, and cycloalkyl;

wherein R₁₁ is H, linear and branched alkyl groups of from 1 to 20 carbon atoms, cycloalkyl, aryl, alkaryl, and O; and

wherein Q and m are as defined above;

and

R₃ and R₄ are independently selected from the group consisting of linear or branched alkyl of from 1 to about 36 carbon atoms, aryl, alkaryl, cycloalkyl, and alkenyl of from 3 to about 36 carbon atoms, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, that Z is not —CH₂CH₂— or —CH₂CH(CH₃)—, and that R₃ and R₄ are not both hydrogen;

(C) R₃ is a cycloalkyl ring independently selected from the group consisting of:

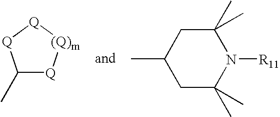

wherein

each Q is C(R₉)(R₁₀), wherein each R₉ and R₁₀ is independently selected from the group consisting of hydrogen, linear and branched alkyl groups of from 1 to 20 carbon atoms, and cycloalkyl;

wherein R₁₁ is H, linear and branched alkyl groups of from 1 to 20 carbon atoms, cycloalkyl, aryl, alkaryl, and O; and wherein Q and m are as defined above;

and

R₂ and R₄ are independently selected from the group consisting of linear or branched alkyl of from 1 to about 36 carbon atoms, aryl, alkaryl, cycloalkyl, and alkenyl of from 3 to about 36 carbon atoms, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, and that Z is not —CH₂CH₂— or —CH₂CH(CH₃)—;

(D) R₂, R₃, and R₄ are independently selected from the group consisting of alkyls of from 3 to about 36 carbon atoms, alkenyls of from 3 to about 36 carbon atoms, aryl, alkaryl, and cycloalkyl, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, that Z is not —CH₂CH₂— or —CH₂CH(CH₃)—; and

(1) at least two of R₂, R₃, and R₄ are independently selected from the group consisting of alkyls of from 3 to about 36 carbon atoms, alkenyls of from 3 to about 36 carbon atoms, wherein said alkyls or alkenyls are branched on the carbon bonded to the phenylenediamine nitrogen; and that

(2) when R₁ is a 2,2,6,6-tetramethylpiperidinyl, R₃ can be hydrogen; and

wherein, when X and Y are ortho or meta to each other, R₂, R₃, and R₄ are in an interrelated relationship selected from the group consisting of:

(E) R₂ is a cycloalkyl ring independently selected from the group consisting of:

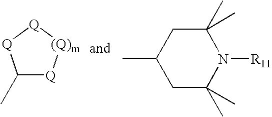

wherein

each Q is C(R₉)(R₁₀), wherein each R₉ and R₁₀ is independently selected from the group consisting of hydrogen, linear and branched alkyl groups of from 1 to 20 carbon atoms, and cycloalkyl;

wherein R₁₁ is H, linear and branched alkyl groups of from 1 to 20 carbon atoms, cycloalkyl, aryl, alkaryl, and O; and wherein Q and m are as defined above;

and

R₃ and R₄ are independently selected from the group consisting of linear or branched alkyl of from 1 to about 36 carbon atoms, aryl, alkaryl, and cycloalkyl, all optionally containing a nitrogen, oxygen, or sulfur atom, hydrogen, alkenyl of from 3 to about 36 carbon atoms, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not —CH₂CH₂— or —CH₂CH(CH₃)—, and that R₃ and R₄ are not both hydrogen;

(F) R₂, R₃, and R₄ are independently selected from the group consisting of linear or branched alkyl of from 1 to about 36 carbon atoms and cycloalkyl, all optionally containing a nitrogen, oxygen, or sulfur atom, hydrogen, aryl, alkaryl, alkenyl of from 3 to about 36 carbon atoms, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not —CH₂CH₂— or —CH₂CH(CH₃)—;

and wherein said phenylenediamine is in the form of a free base, or an oil-soluble salt.

In still another aspect, the present invention is directed the N-cycloalkyl-substituted phenylenediamine additives, per se.

### DESCRIPTION OF THE PREFERRED EMBODIMENTS

In accordance with the present invention, significant deposit-control has been found not only for para-substituted phenylenediamines, but for ortho- and meta-substituted phenylenediamines as well. Phenylenediamines with substitution on the center aromatic ring have also been found to be highly active deposit control agents.

The cycloalkyl substituted phenylenediamines of the invention can also be N-substituted with one or more aryl, alkyl, alkenyl, or alkaryl groups, provided that there is at least one C₅-C₁₂ cycloalkyl group and a total of at least two secondary hydrocarbyl substitutents on the phenylenediamine nitrogens.

Many of the di- and tri-cycloalkyl-substituted phenylenediamine compounds of this invention are novel compositions of matter. It is understood, para-phenylenediamines N-substituted with three or four cycloalkyl groups have not been reported, nor have para-phenylenediamines N-substituted with three cycloalkyl groups and another group, be it substantially alkyl, alkenyl, aryl, or alkaryl in nature. Very few para-phenylenediamines N-substituted with two cycloalkyl groups and one or two other groups, be they substantially alkyl, alkenyl, aryl, or alkaryl in nature, have been reported. Meta-phenylenediamines N-substituted with two cycloalkyl groups and at least one other group, be it substantially alkyl, alkenyl, aryl, or alkaryl in nature, have not been reported. Para-phenylenediamines N-substituted with one cycloalkyl group and two or more other groups attached by a secondary carbon have not been reported. These compounds may be considered to be partially acyclic analogs of the di- and tri-cycloalkyl-substituted phenylenediamines. Diethyl toluenediamines N-substituted with two cycloalkyl groups have not been reported.

The compositions of the present invention comprise a fluid subject to oxidative degradation and an amount of at least one N-cycloalkyl-substituted phenylenediamine additive sufficient to stabilize said fluid against the formation of deposits, wherein the additive is the compound of the invention and has the structure:

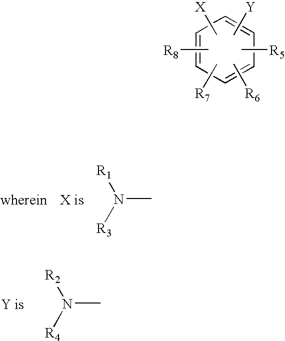

R₁ is a cycloalkyl ring selected from the group consisting of:

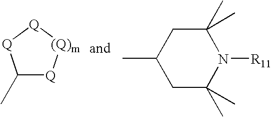

wherein

R₅, R₆, R₇, and R₈ are independently selected from the group consisting of hydrogen, linear and branched alkyl groups of from 1 to 20 carbon atoms, cycloalkyl, allyl, methyallyl, isobutenyl, alkenyl, furfuryl, benzyl, alkaryl, and phenyl;

each Q is C(R₉)(R₁₀), wherein each R₉ and R₁₀ is independently selected from the group consisting of hydrogen, linear and branched alkyl groups of from 1 to 20 carbon atoms, and cycloalkyl;

wherein R₁₁ is H, linear and branched alkyl groups of from 1 to 20 carbon atoms, cycloalkyl, aryl, alkaryl, and O; and wherein Q and m are as defined above;

and

m is an integer of 1 to 8; and

wherein, when X and Y are para to each other, R₂, R₃, and R₄ are in an interrelated relationship selected from the group consisting of:

(A) R₂ is a cycloalkyl ring independently selected from the group consisting of:

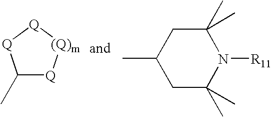

wherein

each Q is C(R₉)(R₁₀), wherein each R₉ and R₁₀ is independently selected from the group consisting of hydrogen, linear and branched alkyl groups of from 1 to 20 carbon atoms, and cycloalkyl;

wherein R₁₁ is H, linear and branched alkyl groups of from 1 to 20 carbon atoms, cycloalkyl, aryl, alkaryl, and O; and wherein Q and m are as defined above;

R₃ is an independently selected cycloalkyl ring of the structure:

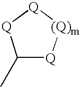

wherein Q and m are as defined above;
and

R₄ is selected from the group consisting of linear or branched alkyl of from 1 to about 36 carbon atoms, aryl, alkaryl, cycloalkyl, and alkenyl of from 3 to about 36 carbon atoms, all optionally containing a nitrogen, oxygen, or sulfur atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not —CH₂CH₂— or —CH₂CH(CH₃)—;

(B) R₂ is a cycloalkyl ring independently selected from the group consisting of:

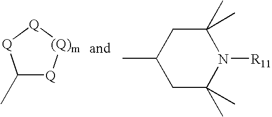

wherein

each Q is C(R₉)(R₁₀), wherein each R₉ and R₁₀ is independently selected from the group consisting of hydrogen, linear and branched alkyl groups of from 1 to 20 carbon atoms, and cycloalkyl;

wherein R₁₁ is H, linear and branched alkyl groups of from 1 to 20 carbon atoms, cycloalkyl, aryl, alkaryl, and O; and

wherein Q and m are as defined above;

and

R₃ and R₄ are independently selected from the group consisting of linear or branched alkyl of from 1 to about 36 carbon atoms, aryl, alkaryl, cycloalkyl, and alkenyl of from 3 to about 36 carbon atoms, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, that Z is not —CH₂CH₂— or —CH₂CH(CH₃)—, and that R₃ and R₄ are not both hydrogen;

(C) R₃ is a cycloalkyl ring independently selected from the group consisting of:

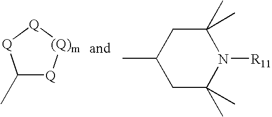

wherein

each Q is C(R₉)(R₁₀), wherein each R₉ and R₁₀ is independently selected from the group consisting of hydrogen, linear and branched alkyl groups of from 1 to 20 carbon atoms, and cycloalkyl;

wherein R₁₁ is H, linear and branched alkyl groups of from 1 to 20 carbon atoms, cycloalkyl, aryl, alkaryl, and O; and wherein Q and m are as defined above;

and

R₂ and R₄ are independently selected from the group consisting of linear or branched alkyl of from 1 to about 36 carbon atoms, aryl, alkaryl, cycloalkyl, and alkenyl of from 3 to about 36 carbon atoms, all optionally containing a nitrogen atom, hydrogen, and ZCO₂_{R}₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, and that Z is not —CH₂CH₂— or —CH₂CH(CH₃)—;

(D) R₂, R₃, and R₄ are independently selected from the group consisting of alkyls of from 3 to about 36 carbon atoms, alkenyls of from 3 to about 36 carbon atoms, aryl, alkaryl, and cycloalkyl, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, that Z is not —CH₂CH₂— or —CH₂CH(CH₃)—; and
(1) at least two of R₂, R₃, and R₄ are independently selected from the group consisting of alkyls of from 3 to about 36 carbon atoms, alkenyls of from 3 to about 36 carbon atoms, wherein said alkyls or alkenyls are branched on the carbon bonded to the phenylenediamine nitrogen; and that
(2) when R₁ is a 2,2,6,6-tetramethylpiperidinyl, R₃ can be hydrogen; and

wherein, when X and Y are ortho or meta to each other, R₂, R₃, and R₄ are in an interrelated relationship selected from the group consisting of:

(E) R₂ is a cycloalkyl ring independently selected from the group consisting of:

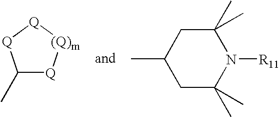

wherein

each Q is C(R₉)(R₁₀), wherein each R₉ and R₁₀ is independently selected from the group consisting of hydrogen, linear and branched alkyl groups of from 1 to 20 carbon atoms, and cycloalkyl;

wherein R₁₁ is H, linear and branched alkyl groups of from 1 to 20 carbon atoms, cycloalkyl, aryl, alkaryl, and O; and wherein Q and m are as defined above;

and

R₃ and R₄ are independently selected from the group consisting of linear or branched alkyl of from 1 to about 36 carbon atoms, aryl, alkaryl, and cycloalkyl, all optionally containing a nitrogen, oxygen, or sulfur atom, hydrogen, alkenyl of from 3 to about 36 carbon atoms, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S;provided that Z is not —CH₂CH₂— or —CH₂CH(CH₃)—, and that R₃ and R₄ are not both hydrogen;

(F) R₂, R₃, and R₄ are independently selected from the group consisting of linear or branched alkyl of from 1 to about 36 carbon atoms and cycloalkyl, all optionally containing a nitrogen, oxygen, or sulfur atom, hydrogen, aryl, alkaryl, alkenyl of from 3 to about 36 carbon atoms, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not —CH₂CH₂— or —CH₂CH(CH₃)—; and wherein said phenylenediamine is in the form of a free base, or an oil-soluble salt.

Preferred are compounds of the above formulas wherein:

R₁ and R₂ are independently selected from cyclopentyl, cyclohexyl cycloheptyl and cyclooctyl; and R₉-R₁₁ are independently linear or branched alkyl of from one to about eight carbons, or hydrogen;

where R₃ is independently selected from R₁, R₂, linear or branched alkyl of from 1 to about 18 carbons, cyclopentyl, cyclohexyl, cycloheptyl and cyclooctyl, phenyl, tolyl, alkylphenyl, benzyl, alkenyl of 5 to about 16 carbons, or hydrogen;

where R₄ is independently selected from R₁, R₂, R₃, linear or branched alkyl of from 1 to about 18 carbons, cyclopentyl, cyclohexyl, cycloheptyl and cyclooctyl, phenyl, tolyl, alkylphenyl, benzyl, of 3 to 36 carbons, but preferably 3 to about 16 carbons, or hydrogen;

and R₅-R₈ are independently selected from linear or branched alkyl of 1 to about 6 carbons, or hydrogen;

or R₁ and R₂ are as defined above, and R₃ and R₄ are independently selected from linear or branched alkyl of from 1 to about 18 carbons, cyclopentyl, cyclohexyl, cycloheptyl cyclooctyl, phenyl, tolyl, alkylphenyl, benzyl, allyl, alkenyl, or H; and R₉-R₁₁ are independently selected from linear or branched alkyl of 1 to about 6 carbons, or hydrogen; and wherein said phenylenediamine is in the form of a free base, or an oil-soluble salt.

More preferred are compounds of the above formulas wherein:

R₁ is selected from cyclopentyl or cyclohexyl, and R₉-R₁₁ are independently linear or branched alkyl of from 1 to about 4 carbons, or hydrogen; and

R₂ is selected from R₁, cyclopentyl, cyclohexyl (wherein the substituents are independently linear or branched alkyl of from 1 to about 8 carbons, or hydrogen); phenyl, tolyl, alkylphenyl, linear or branched alkyl of from 1 to about 12 carbons, benzyl, and

where R₃ is independently selected from R₁, R₂, linear or branched alkyl of from 1 to about 10 carbons, phenyl, tolyl, alkylphenyl;

where R₄ is independently selected from R₁, R₂, R₃, linear or branched alkyl of from 1 to about 12 carbons, cyclopentyl, cyclohexyl, phenyl, tolyl, alkylphenyl, benzyl, alkenyl, or hydrogen; and

R₅-R₈ are independently selected from linear or branched alkyl of 1 to about 4 carbons, or hydrogen; or

R₁ and R₂ are as defined above, and R₃ and R₄ are independently selected from R₁ and R₂, cyclopentyl, cyclohexyl, linear or branched alkyl of from 1 to about 12 carbons, phenyl, tolyl, alkylphenyl, benzyl, alkenyl, or hydrogen; and R₁₁-R₁₈ are independently selected from linear or branched alkyl of 1 to about 4 carbons, or hydrogen; and

wherein said phenylenediamine is in the form of a free base, or an oil-soluble salt.

A desirable compound includes an N-cycloalkyl substituted phenylenediamine sufficient to stabilize the fluid against the formation of deposits. Such an additive has the structure:

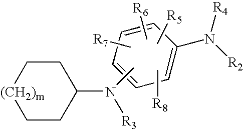

where m is 0, 1, 2, or 3, and R₂, R₃, and R₄ are independently H, alkyl, branched alkyl, cycloalkyl, alkenyl of 3 to 36 carbons, but preferably 3 to about 16 carbons, alkaryl, benzyl, phenyl, or aryl, optionally containing an N, O, or S atom; and R₅, R₆ R₇, and R₈ are independently selected from linear or branched alkyl of 1 to about 20 carbons, or hydrogen; with the proviso that at least two of R₂, R₃, and R₄ are independently alkyls branched on the carbon bonded to the phenylenediamine nitrogen.

Another desirable compound includes an N,N′-dicycloalkyl substituted phenylenediamine sufficient to stabilize the fluid against the formation of deposits. Such an additive has the structure:

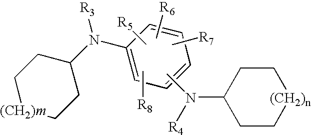

where m and n are independently 0, 1, 2, or 3, and R₃ and R₄ are independently H, alkyl, branched alkyl, cycloalkyl, alkenyl of 3 to 36 carbons, but preferably 5 to about 16 carbons, alkaryl, benzyl, phenyl, or aryl, optionally containing an N, O, or S atom; and R₅, R₆, R₇, and R₈ are independently selected from linear or branched alkyl of 1 to about 20 carbons, or hydrogen.

Yet another desirable compound includes an N,N,N′-tricycloalkyl substituted phenylenediamines sufficient to stabilize the fluid against the formation of deposits. Such an additive has the structure:

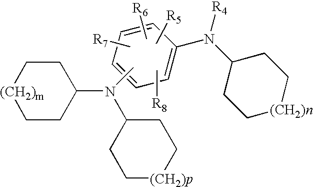

where m, n and p are independently 0, 1, 2, or 3, and R₄ is independently alkyl, branched alkyl, cycloalkyl, allyl, alkenyl, alkaryl, benzyl, phenyl, or aryl, optionally containing an N, O, or S atom; and R₅, R₆, R₇ and R₈ are independently selected from linear or branched alkyl organic materials can be natural or synthetic. These organic materials can include “functional fluids,” lubricating oils, greases, and fuels, as well as automatic and manual transmission fluids, power steering fluid, hydraulic fluids, gas turbine oils, compressor lubricants, automotive and industrial gear lubricants, and heat transfer oils. Lubricating oil compositions useful in the practice of the present invention comprise a major amount of oil of lubricating viscosity and a minor amount of at least one phenylenediamine compound having one or more cycloalkyl substituents on each nitrogen atom.

Oils of lubricating viscosity useful in the context of the present invention can be selected from natural lubricating oils, synthetic lubricating oils, and mixtures thereof. The lubricating oil can range in viscosity from light distillate mineral oils to heavy lubricating oils, such as gasoline engine oils, mineral lubricating oils, and heavy duty diesel oils. Generally, the viscosity of the oil ranges from about 2 centistokes to about 40 centistokes, especially from about 4 centistokes to about 20 centistokes, as measured at 100° C.

The diesel fuel is a petroleum-based fuel oil, especially a middle distillate fuel oil. Such distillate fuel oils generally boil within the range of from 110° C. to 500° C., e.g. 150° C. to 400° C. The fuel oil may comprise atmospheric distillate or vacuum distillate, cracked gas oil, or a blend in any proportion of straight run and thermally and/or refinery streams such as catalytically cracked and hydro-cracked distillates.

Other examples of diesel fuels include Fischer-Tropsch fuels. Fischer-Tropsch fuels, also known as FT fuels, include those described as gas-to-liquid (GTL) fuels, biomass-to-liquid (BTL) fuels and coal conversion fuels. To make such fuels, syngas (CO +H₂) is first generated and then converted to normal paraffins by a Fischer-Tropsch process. The normal paraffins can then be modified by processes such as catalytic cracking/reforming or isomerization, hydrocracking and hydroisomerization to yield a variety of hydrocarbons such as iso-paraffins, cyclo-paraffins and aromatic compounds. The resulting FT fuel can be used of 1 to about 20 carbons, or hydrogen.

Preferably, the phenylenediamine compound has, or has on average, a nitrogen content of from about 4 weight % to about 14 weight %, preferably from about 5 weight % to about 11 weight %, more preferably from about 5.5 weight % to about 10.5 weight %.

Particularly preferred N-cycloalkyl-substituted phenylenediamine additives for use in the practice of the present invention include but are not limited to:
N-(1-methyldecyl)-N′-(2,2,6,6-tetramethyl-4-piperdinyl)-p-phenylenediamine;
N-cyclohexyl-N-phenyl-o-phenylenediamine;
N-cyclohexyl-N′-phenyl-o-phenylenediamine;
N,N′-dicyclohexyl-m-phenylenediamine;
N,N′-dicyclohexyl-m-toluenediamine;
N,N′-dicyclohexyl-3,5-diethyl-2,4-toluenediamine;
N,N′-dicyclohexyl-3,5-diethyl-2,6-toluenediamine;
N-cyclohexyl-N-(2-ethylhexyl)-N′-phenyl-p-phenylenediamine;
N-cyclohexyl-N,N′-di(sec-butyl)-p-phenylenediamine;
N,N′-dicyclooctyl-N-ethyl-p-phenylenediamine;
N,N′-dicyclohexyl-N-isopropyl-p-phenylenediamine;
N,N′-dicyclohexyl-N-isobutyl-p-phenylenediamine;
N,N′-dicyclohexyl-N-butyl-m-phenylenediamine;
N,N′-dicyclohexyl-N-butyl-p-phenylenediamine;
N,N′-dicyclohexyl-N-isobutyl-m-phenylenediamine;
N,N′-dicyclohexyl-N-isobutyl-m-phenylenediamine;
N,N′-dicyclohexyl-N-heptyl-m-phenylenediamine;
N,N′-dicyclohexyl-N-heptyl-p-phenylenediamine;
N,N′-dicyclohexyl-N′-phenyl-p-phenylenediamine;
N,N-dicyclohexyl-N′-phenyl-p-phenylenediamine;
N,N′-dicyclohexyl-N,N′-difurfuryl-p-phenylenediamine;
N,N′-dicyclohexyl-N,N′-dibenzyl-p-phenylenediamine;
N,N′-dicyclohexyl-N′-phenyl-N-heptyl-p-phenylenediamine;
N,N′-dicyclohexyl-N,N′-diethyl-p-phenylenediamine;
N,N′-dicyclohexyl-N,N′-dibutyl-m-phenylenediamine;
N,N′-dicyclohexyl-N,N′-dibutyl-p-phenylenediamine;
N,N′-dicyclohexyl-N,N′-diisobutyl-m-phenylenediamine;
N,N′-dicyclohexyl-N,N′-diisobutyl-p-phenylenediamine;
N,N′-dicyclohexyl-N,N′-diheptyl-m-phenylenediamine;
N,N′-dicyclohexyl-N,N′-diheptyl-p-phenylenediamine;
N,N′-dicyclooctyl-N,N′-diethyl-p-phenylenediamine;
N,N,N′-tricyclopentyl-p-phenylenediamine;
N,N,N′-tricyclohexyl-p-phenylenediamine;
N,N,N′-tricyclohexyl-N′-heptyl-p-phenylenediamine;
N,N,N′-tricyclohexyl-N′-propyl-p-phenylenediamine;
N,N,N′-tricyclohexyl-N′-butyl-p-phenylenediamine;
N-(2-ethylhexyl)-N,N′,N′-tricyclohexyl-p-phenylenediamine;
N,N,N′-tricyclohexyl-N′-phenyl-p-phenylenediamine;
N,N,N′,N′-tetracyclohexyl-p-phenylenediamine;
N,N′-bis(2-decalinyl)-p-phenylenediamine;
N,N′-dicyclohexyl-4,6-diisobutyl-1,3-phenylenediamine; and
N,N′-dicyclohexyl-4-isobutenyl-6-isobutyl-1,3-phenylenediamine.

The compounds of the present invention improve the oxidative stability of organic materials, which are subject to oxidative, thermal, and/or light-induced degradation. These as such or in combination with other fuel components and fuel types. Also suitable are diesel fuels derived from plant or animal sources. These can be used alone or in combination with other types of fuel.

Preferably, the diesel fuel has a sulfur content of at most 0.05% by weight, more preferably of at most 0.035% by weight, especially of at most 0.015%. Fuels with even lower levels of sulfur are also suitable, such as fuels with less than 50 ppm sulfur by weight, preferably less than 20 ppm, for example, 10 ppm or less.

Oils and fats derived from plant or animal materials are increasingly finding application as fuels and, in particular, as partial or complete replacements for petroleum derived middle distillate fuels such as diesel. Commonly, such fuels are known as “biofuels” or “biodiesels.” Biofuels may be derived from many sources. Among the most common are the alkyl, often methyl, esters of fatty acids extracted from plants, such as rapeseed, sunflower, and the like. These types of fuel are often referred to as FAME (fatty acid methyl esters).

Natural oils include animal oils and vegetable oils (e.g., lard oil, castor oil); liquid petroleum oils and hydrorefined, solvent-treated or acid-treated mineral oils of the paraffinic, naphthenic, and mixed paraffinic-naphthenic types. Oils of lubricating viscosity derived from coal or shale also serve as useful base oils. Other examples of oils and fats derived from animal or vegetable material are rapeseed oil, coriander oil, soya bean oil, cottonseed oil, sunflower oil, castor oil, olive oil, peanut oil, maize oil, almond oil, palm kernel oil, coconut oil, mustard seed oil, jatropha oil, beef tallow, and fish oils. Further examples include oils derived from corn, jute, sesame, shea nut, ground nut, and linseed oil, and may be derived therefrom by methods known in the art. Rapeseed oil, which is a mixture of fatty acids partially esterified with glycerol, is available in large quantities and can be obtained in a simple way by pressing from rapeseed. Recycled oils such as used kitchen oils are also suitable.

Useful examples of alkyl esters of fatty acids can include commercial mixtures of the ethyl, propyl, butyl and especially methyl esters of fatty acids with 12 to 22 carbon atoms. For example, lauric acid, myristic acid, palmitic acid, palmitoleic acid, stearic acid, oleic acid, elaidic acid, petroselic acid, ricinoleic acid, elaeostearic acid, linoleic acid, linolenic acid, eicosanoic acid, gadoleic acid, docosanoic acid, or erucic acid are useful and have an iodine number from 50 to 150, especially 90 to 125. Mixtures with particularly advantageous properties are those which contain mainly, i.e., at least 50 weight %, methyl esters of fatty acids with 16 to 22 carbon atoms and 1, 2, or 3 double bonds. The preferred lower alkyl esters of fatty acids are the methyl esters of oleic acid, linoleic acid, linolenic acid, and erucic acid.

Commercial mixtures of the stated kind are obtained for example by cleavage and esterification of animal and vegetable fats and oils by their transesterification with lower aliphatic alcohols. For production of alkyl esters of fatty acids, it is advantageous to start from fats and oils which contain low levels of saturated acids, less than 20%, and which have an iodine number of less than 130. Blends of the following esters or oils are suitable, e.g., rapeseed, sunflower, coriander, castor, soya bean, peanut, cotton seed, beef tallow, and the like. Alkyl esters of fatty acids based on a new variety of rapeseed oil, the fatty acid component of which comprises more than 80 weight % unsaturated fatty acids with 18 carbon atoms, are preferred.

Particularly preferred are oils capable of being utilized as biofuels. Biofuels, i.e., fuels derived from animal or vegetable material, are believed to be less damaging to the environment on combustion and are obtained from a renewable source. It has been reported that on combustion less carbon dioxide is formed by the equivalent quantity of petroleum distillate fuel, e.g., diesel fuel, and very little sulfur dioxide is formed. Certain derivatives of vegetable oil, e.g., those obtained by saponification and re-esterification with a monohydric alkyl alcohol, can be used as a substitute for diesel fuel.

Preferred biofuels are vegetable oil derivatives, of which particularly preferred biofuels are alkyl ester derivatives of rapeseed oil, cottonseed oil, soya bean oil, sunflower oil, olive oil, or palm oil, rapeseed oil methyl ester being especially preferred, either alone or in admixture with other vegetable oil derivatives, e.g., mixtures in any proportion of rapeseed oil methyl ester and palm oil methyl ester.

At present, biofuels are most commonly used in combination with petroleum-derived oils. The present invention is applicable to mixtures of biofuel and petroleum-derived fuels in any ratio. For example, at least 5%, preferably at least 25%, more preferably at least 50%, and most preferably at least 95% by weight of the oil, may be derived from a plant or animal source.

Synthetic lubricating oils include hydrocarbon oils and halo-substituted hydrocarbon oils such as polymerized and interpolymerized olefins (e.g., polybutylenes, polypropyleres, propylene-isobutylene copolymers, chlorinated polybutylenes, poly(1-hexenes), poly(1-octenes), poly(1-decenes)); alkylbenzenes (e.g., dodecylbenzenes, tetradecylbenzenes, dinonylbenzenes, di(2-ethylhexyl)benzenes); polyphenyls (e.g., biphenyls, terphenyls, alkylated polyphenols); and alkylated diphenyl ethers and alkylated diphenyl sulfides and derivative, analogs, and homologs thereof. Also useful are synthetic oils derived from a gas to liquid process from Fischer-Tropsch synthesized hydrocarbons, which are commonly referred to as gas to liquid or “GTL” base oils.

Alkylene oxide polymers and interpolymers and derivatives thereof where the terminal hydroxyl groups have been modified by esterification, etherification, etc., constitute another class of known synthetic lubricating oils. These are exemplified by polyoxyalkylene polymers prepared by polymerization of ethylene oxide or propylene oxide and the alkyl and aryl ethers of polyoxyalkylene polymers (e.g., methyl-polyisopropylene glycol ether having a molecular weight of 1000 or diphenyl ether of polyethylene glycol having a molecular weight of 1000 to 1500), and mono- and polycarboxylic esters thereof, for example, the acetic acid esters, mixed C₃-C₈ fatty acid esters, and C₁₃ oxo acid diester of tetraethylene glycol.

Another suitable class of synthetic lubricating oils comprises the esters of dicarboxylic acids (e.g., phthalic acid, succinic acid, alkyl succinic acids and alkenyl succinic acids, maleic acid, azelaic acid, suberic acid, sebasic acid, fumaric acid, adipic acid, linoleic acid dimer, malonic acid, alkylmalonic acids, alkenyl malonic acids) with a variety of alcohols (e.g., butyl alcohol, hexyl alcohol, dodecyl alcohol, 2-ethylhexyl alcohol, ethylene glycol, diethylene glycol monoether, propylene glycol). Specific examples of such esters includes dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, the 2-ethylhexyl diester of linoleic acid dimer, and the complex ester formed by reacting one mole of sebacic acid with two moles of tetraethylene glycol and two moles of 2-ethylhexanoic acid.

Esters useful as synthetic oils also include those made from C₅ to C₁₂ monocarboxylic acids and polyols and polyol esters such as neopentyl glycol, trimethylolpropane, pentaerythritol, dipentaerythritol and tripentaerythritol.

Silicon-based oils such as the polyalkyl-, polyaryl-, polyalkoxy- or polyaryloxysilicone oils and silicate oils comprise another useful class of synthetic lubricants; such oils include tetraethyl silicate, tetraisopropyl silicate, tetra-(2-ethylhexyl)silicate, tetra-(4-methyl-2-ethylhexyl)silicate, tetra-(p-tert-butyl-phenyl) silicate, hexa-(4-methyl-2-ethylhexyl)disiloxane, poly(methyl)siloxanes and poly(methylphenyl)siloxanes. Other synthetic lubricating oils include liquid esters of phosphorus-containing acids (e.g., tricresyl phosphate, trioctyl phosphate, diethyl ester of decylphosphonic acid) and polymeric tetrahydrofurans.

The oil of lubricating viscosity can comprise a Group I, Group II, or Group III base stock or base oil blends of the aforementioned base stocks. Preferably, the oil of lubricating viscosity is a Group II or Group III base stock, or a mixture thereof, or a mixture of a Group I base stock and one or more of a Group II and Group III. Preferably, a major amount of the oil of lubricating viscosity is a Group II, Group III, Group IV, or Group V base stock, or a mixture thereof. The base stock, or base stock blend, preferably has a saturate content of at least 65%, more preferably at least 75%, such as at least 85%. Most preferably, the base stock, or base stock blend, has a saturate content of greater than 90%. Preferably, the oil or oil blend will have a sulfur content of less than 1%, preferably less than 0.6%, and most preferably less than 0.4%, by weight.

Preferably the volatility of the oil or oil blend, as measured by the Noack volatility test (ASTM D5880), is less than or equal to 30%, preferably less than or equal to 25%, more preferably less than or equal to 20%, most preferably less than or equal to 16%. Preferably, the viscosity index (VI) of the oil or oil blend is at least 85, preferably at least 100, most preferably from about 105 to 140.

Definitions for the base stocks and base oils in this invention are the same as those found in the American Petroleum Institute (API) publication “Engine Oil Licensing and Certification System,” Industry Services Department (14th ed., December 1996), Addendum 1, December 1998. This publication categorizes base stocks as follows.

(a) Group I base stocks contain less than 90 percent saturates and/or greater than 0.03 percent sulfur and have a viscosity index greater than or equal to 80 and less than 120 using the test methods specified in Table 1.

(b) Group II base stocks contain greater than or equal to 90 percent saturates and less than or equal to 0.03 percent sulfur and have a viscosity index greater than or equal to 80 and less than 120 using the test methods specified in Table 1.

(c) Group III base stocks contain greater than or equal to 90 percent saturates and less than or equal to 0.03 percent sulfur and have a viscosity index greater than or equal to 120 using the test methods specified in Table 1.

(d) Group IV base stocks are polyalphaolefins (PAO).

(e) Group V base stocks include all other base stocks not included in Groups I, II, III, or IV.

TABLE 1
Analytical Methods for Base Stock
Property Test Method

| | |
|---|---|
| Saturates | ASTM D 2007 |
| Viscosity Index | ASTM D 2270 |
| Sulfur | ASTM D 2622 |
| | ASTM D 4294 |
| | ASTM D 4927 |
| | ASTM D 3120 |

Additional additives may be incorporated in the compositions of the invention to enable them to meet particular requirements. Examples of additives that may be included in the lubricating oil compositions are dispersants, detergents, metal rust inhibitors, viscosity index improvers, corrosion inhibitors, oxidation inhibitors, friction modifiers, other dispersants, anti-foaming agents, anti-wear agents and pour point depressants. Some are discussed in further detail below.

Lubricating oil compositions of the present invention can further contain one or more ashless dispersants, which effectively reduce formation of deposits upon use in gasoline and diesel engines, when added to lubricating,oils. Ashless dispersants useful in the compositions of the present invention comprise an oil soluble polymeric long chain backbone having functional groups capable of associating with particles to be dispersed. Typically, such dispersants comprise amine, alcohol, amide or ester polar moieties attached to the polymer backbone, often via a bridging group. The ashless dispersant can be, for example, selected from oil soluble salts, esters, amino-esters, amides, imides, and oxazolines of long chain hydrocarbon-substituted mono- and polycarboxylic acids or anhydrides thereof; thiocarboxylate derivatives of long chain hydrocarbons; long chain aliphatic hydrocarbons having polyamine moieties attached directly thereto; and Mannich condensation products formed by condensing a long chain substituted phenol with formaldehyde and polyalkylene polyamine.

Preferred dispersants include polyamine-derivatized poly alpha-olefin, dispersants, particularly ethylene/butene alpha-olefin and polyisobutylene-based dispersants. Particularly preferred are ashless dispersants derived from polyisobutylene substituted with succinic anhydride groups and reacted with polyethylene amines, e.g., polyethylene diamine, tetraethylene pentamine; or a polyoxyalkylene polyamine, e.g., polyoxypropylene diamine, trimethylolaminomethane; a hydroxy compound, e.g., pentaerythritol; and combinations thereof. One particularly preferred dispersant combination is a combination of (A) polyisobutylene substituted with succinic anhydride groups and reacted with (B) a hydroxy compound, e.g., pentaerythritol; (C) a polyoxyalkylene polyamine, e.g., polyoxypropylene diamine, or (D) a polyalkylene diamine, e.g., polyethylene diamine and tetraethylene pentamine using about 0.3 to about 2 moles of (B), (C) and/or (D) per mole of (A). Another preferred dispersant combination comprises a combination of (A) polyisobutenyl succinic anhydride with (B) a polyalkylene polyamine, e.g., tetraethylene pentamine, and (C) a polyhydric alcohol or polyhydroxy-substituted aliphatic primary amine, e.g., pentaerythritol or trismethylolaminomethane, as described in U.S. Pat. No. 3,632,51 1.

Another class of ashless dispersants, comprises Mannich base condensation products. Generally, these products are prepared by condensing about one mole of an alkyl-substituted mono- or polyhydroxy benzene with about 1 to 2.5 moles of carbonyl compound(s) (e.g., formaldehyde and paraformaldehyde) and about 0.5 to 2 moles of polyalkylene polyamine, as disclosed, for example, in U.S. Pat. No. 3,442,808. Such Mannich base condensation products can include a polymer product of a metallocene catalyzed polymerization as a substituent on the benzene group, or can be reacted with a compound containing such a polymer substituted on a succinic anhydride,in a manner similar to that described in U.S. Pat. No. 3,442,808. Examples of functionalized and/or derivatized olefin polymers synthesized using metallocene catalyst systems are described in the publications identified supra.

The dispersant can be further post treated by a variety of conventional post treatments such as boration, as generally taught in U.S. Pat. Nos. 3,087,936 and 3,254,025. Boration of the dispersant is readily accomplished by treating an acyl nitrogen-containing dispersant with a boron compound, such as boron oxide, boron halide boron acids, and esters of boron acids, in an amount sufficient to provide from about 0.1 to about 20 atomic proportions of boron for each mole of acylated nitrogen composition. Useful dispersants contain from about 0.05 to about 2.0 weight %, e.g., from about 0.05 to about 0.7 weight % boron. The boron, which appears in the product as dehydrated boric acid polymers (primarily (HBO₂)₃), is believed to attach to the dispersant imides and diimides as amine salts, e.g., the metaborate salt of the diimide. Boration can be performed by adding from about 0.5 to 4 weight %, e.g., from about 1 to about 3 weight % (based on the mass of acyl nitrogen compound) of a boron compound, preferably boric acid, usually as a slurry, to the acyl nitrogen compound and heating with stirring at from about 135° C. to about 190° C., e.g., 140° C. to 170° C., for from about one to about five hours, followed by nitrogen stripping. Alternatively, the boron treatment can be conducted by adding boric acid to a hot reaction mixture of the dicarboxylic acid material and amine, while removing water. Other post reaction processes commonly known in the art can also be applied.

The dispersant can also be further post treated by reaction with a so-called “capping agent.” Conventionally, nitrogen-containing dispersants have been “capped” to reduce the adverse effect such dispersants have on the fluoroelastomer engine seals. Numerous capping agents and methods are known. Of the known “capping agents,” those that convert basic dispersant amino groups to non-basic moieties (e.g., amido or imido groups) are most suitable. The reaction of a nitrogen-containing dispersant and alkyl acetoacetate (e.g., ethyl acetoacetate (EAA)) is described, for example, in U.S. Pat. Nos. 4,839,071, 4,839,072, and 4,579,675. The reaction of a nitrogen-containing dispersant and formic acid is described, for example, in U.S. Pat. No. 3,185,704. The reaction product of a nitrogen-containing dispersant and other suitable capping agents are described in U.S. Pat. No. 4,663,064 (glycolic acid); U.S. Pat. Nos. 4,612,132, 5,334,321, 5,356,552, 5,716,912, 5,849,676, and 5,861,363 (alkyl and alkylene carbonates, e.g., ethylene carbonate); U.S. Pat. No. 5,328,622 (mono-epoxide); U.S. Pat. No. 5,026,495; U.S. Pat. Nos. 5,085,788, 5,259,906, 5,407,591 (poly (e.g., bis)-epoxides); and U.S. Pat. No. 4,686,054 (maleic anhydride or succinic anhydride). The foregoing list is not exhaustive, and other methods of capping nitrogen-containing dispersants are known to those skilled in the art.

For adequate piston deposit control, a nitrogen-containing dispersant can be added in an amount providing the lubricating oil composition with from about 0.03 weight % to about 0.15 weight %, preferably from about 0.07 to about 0.12 weight %, of nitrogen.

Metal-containing or ash-forming detergents function both as detergents to reduce or remove deposits and as acid neutralizers or rust inhibitors, thereby reducing wear and corrosion and extending engine life. Detergents generally comprise a polar head with a long hydrophobic tail, with the polar head comprising a metal salt of an acidic organic compound. The salts can contain a substantially stoichiometric amount of the metal, in which case they are usually described as normal or neutral salts, and would typically have a total base number or TBN (as can be measured by ASTM D2896) of from 0 to 80. A large amount of a metal base can be incorporated by reacting excess metal compound (e.g., an oxide or hydroxide) with an acidic gas (e.g., carbon dioxide). The resulting overbased detergent comprises neutralized detergent as the outer layer of a metal base (e.g. carbonate) micelle. Such overbased detergents can have a TBN of 150 or greater and typically will have a TBN of from 250 to 450 or more.

Detergents that can be used include oil-soluble neutral and overbased sulfonates, phenates, sulfurized phenates, thiophosphonates, salicylates, naphthenates, and other oil-soluble carboxylates of a metal, particularly the alkali or alkaline earth metals, e.g., sodium, potassium, lithium, calcium, and magnesium. The most commonly used metals are calcium and magnesium, which can both be present in detergents used in a lubricant, and mixtures of calcium and/or magnesium with sodium. Particularly convenient metal detergents are neutral and overbased calcium sulfonates having TBN of from 20 to 450 TBN, and neutral and overbased calcium phenates and sulfurized phenates having TBN of from 50 to 450. Combinations of detergents, whether overbased or neutral or both, can be used.

Sulfonates can be prepared from sulfonic acids which are typically obtained by the sulfonation of alkyl substituted aromatic hydrocarbons such as those obtained from the fractionation of petroleum or by the alkylation of aromatic hydrocarbons. Examples included those obtained by alkylating benzene, toluene, xylene, naphthalene, diphenyl, or their halogen derivatives such as chlorobenzene, chlorotoluene and chloronaphthalene. The alkylation can be performed in the presence of a catalyst with alkylating agents having from about 3 to more than 70 carbon atoms. The alkaryl sulfonates usually contain from about 9 to about 80 or more carbon atoms, preferably from about 16 to about 60 carbon atoms, per alkyl substituted aromatic moiety.

The oil soluble sulfonates or alkaryl sulfonic acids can be neutralized with oxides, hydroxides, alkoxides, carbonates, carboxylate, sulfides, hydrosulfides, nitrates, borates, and ethers of the metal. The amount of metal compound is chosen having regard to the desired TBN of the final product but typically ranges from about 100 to 220 weight % (preferably at least 125 weight %) of that stoichiometrically required.

Metal salts of phenols and sulfurized phenols are prepared by reaction with an appropriate metal compound such as an oxide or hydroxide, and neutral or overbased products can be obtained by methods well known in the art. Sulfurized phenols can be prepared by reacting a phenol with sulfur or a sulfur containing compound such as hydrogen sulfide, sulfur monohalide, or sulfur dihalide, to form products which are generally mixtures of compounds in which two or more phenols are bridged by sulfur containing bridges.

Dihydrocarbyl dithiophosphate metal salts are frequently used as antiwear and antioxidant agents. The metal can be an alkali or alkaline earth metal, or aluminum, lead, tin, molybdenum, manganese, nickel or copper. The zinc salts are most commonly used in lubricating oil in amounts of 0.1 to 10 weight %, preferably 0.2 to 2 weight %, based upon the total weight of the lubricating oil composition. They can be prepared in accordance with known techniques by first forming a dihydrocarbyl dithiophosphoric acid (DDPA), usually by reaction of one or more alcohols or a phenol with P₂S₅ and then neutralizing the formed DDPA with a zinc compound. For example, a dithiophosphoric acid can be made by reacting mixtures of primary and secondary alcohols. Alternatively, multiple dithiophosphoric acids can be prepared where the hydrocarbyl groups on one are entirely secondary in character and the hydrocarbyl groups on the others are entirely primary in character. To make the zinc salt, any basic or neutral zinc compound could be used, but the oxides, hydroxides, and carbonates are most generally employed. Commercial additives frequently contain an excess of zinc due to the use of an excess of the basic zinc compound in the neutralization reaction.

The preferred zinc dihydrocarbyl, dithiophosphates are oil soluble salts of dihydrocarbyl dithiophosphoric acids and can comprise zinc dialkyl dithiophosphates. The present invention can be particularly useful when used with passenger car diesel engine lubricant compositions containing phosphorus levels of from about 0.02 to about 0.12 weight %, such as from about 0.03 to about 0.10 weight %, or from about 0.05 to about 0.08 weight %, based on the total mass of the composition and heavy duty diesel engine lubricant compositions containing phosphorus levels of from about 0.02 to about 0.16 weight %, such as from about 0.05 to about 0.14 weight %, or from about 0.08 to about 0.12 weight %, based on the total mass of the composition. In one preferred embodiment, lubricating oil compositions of the present invention contain zinc dialkyl dithiophosphate derived predominantly (e.g., over 50 mol. %, such as over 60 mol. %) from secondary alcohols.

Oxidation inhibitors or antioxidants reduce the tendency of mineral oils to deteriorate in service. Oxidative deterioration can be evidenced by sludge in the lubricant, varnish-like deposits on the metal surfaces, and by viscosity growth. Such oxidation inhibitors include hindered phenols, alkaline earth metal salts of alkylphenolthioesters having preferably C₅ to C₁₂ alkyl side chains, calcium nonylphenol sulfide, oil soluble phenates and sulfurized phenates, phosphosulfurized or sulfurized hydrocarbons, phosphorous esters, metal thiocarbamates, oil soluble copper compounds as described in U.S. Pat. No. 4,867,890, and molybdenum-containing compounds.

Typical oil soluble aromatic amines having at least two aromatic groups attached directly to one amine nitrogen contain from 6 to 16 carbon atoms. The amines can contain more than two aromatic groups. Compounds having a total of at least three aromatic groups, in which two aromatic groups are linked by a covalent bond or by an atom or group (e.g., an oxygen or sulfur atom, or a —CO—, —SO₂— or alkylene group) and two are directly attached to one amine nitrogen, are also considered aromatic amines having at least two aromatic groups attached directly to the nitrogen. The aromatic rings are typically substituted by one or more substituents selected from alkyl, cycloalkyl, alkoxy, aryloxy, acyl, acylamino, hydroxy, and nitro groups.

Multiple antioxidants are commonly employed in combination. In one preferred embodiment, lubricating oil compositions of the present invention, in addition to the phenylenediamine compound(s) added to ameliorate soot-induced viscosity increase, contain from about 0.1 to about 1.2 weight % of aminic antioxidant and from about 0.1 to about 3 weight % of phenolic antioxidant. In another preferred embodiment, lubricating oil compositions of the present invention contain from about 0.1 to about 1.2 weight % of aminic antioxidant, from about 0.1 to about 3 weight % of phenolic antioxidant and a molybdenum compound in an amount providing the lubricating oil composition from about 10 to about 1000 ppm of molybdenum. Preferably, lubricating oil compositions useful in the practice of the present invention, particularly lubricating oil compositions useful in the practice of the present invention that are required to contain no greater than 1200 ppm of phosphorus, contain ashless antioxidants other than phenylenediamines, in an amount of from about 0.1 to about 5 weight %, preferably from about 0.3 weight % to about 4 weight %, more preferably from about 0.5 weight % to about 3 weight %. Where the phosphorus content is required to be lower, the amount of ashless antioxidant other than phenylenediamine will preferably increase accordingly.

Representative examples of suitable viscosity modifiers are polyisobutylene, copolymers of ethylene and propylene, polymethacrylates, methacrylate copolymers, copolymers of an unsaturated dicarboxylic acid and a vinyl compound, interpolymers of styrene and acrylic esters, and partially hydrogenated copolymers of styrene/isoprene, styrenelbutadiene, and isoprene/butadiene, as well as the partially hydrogenated homopolymers of butadiene and isoprene.

A viscosity index improver dispersant functions both as a viscosity index improver and as a dispersant. Examples of viscosity index improver dispersants include reaction products of amines, for example, polyamines, with a hydrocarbyl-substituted mono- or dicarboxylic acid in which the hydrocarbyl substituent comprises a chain of sufficient length to impart viscosity index improving properties to the compounds. In general, the viscosity index improver dispersant can be, for example, a polymer of a C₄ to C₂₄ unsaturated ester of vinyl alcohol or a C₃ to C₁₀ unsaturated mono-carboxylic acid or a C₄ to C₁₀ di-carboxylic acid with an unsaturated nitrogen-containing monomer having 4 to 20 carbon atoms; a polymer of a C₂ to C₂₀ olefin with an unsaturated C₃ to C₁₀ mono- or di-carboxylic acid neutralized with an amine, hydroxyamine or an alcohol; or a polymer of ethylene with a C₃ to C₂₀ olefin further reacted either by grafting a C₄ to C₂₀ unsaturated nitrogen-containing monomer thereon or by grafting an unsaturated acid onto the polymer backbone and then reacting carboxylic acid groups of the grafted acid with an amine, hydroxy amine, or alcohol.

Friction modifiers and fuel economy agents that are compatible with the other ingredients of the final oil can also be included. Examples of such materials include glyceryl monoesters of higher fatty acids, for example, glyceryl mono-oleate; esters of long chain polycarboxylic acids with diols, for example, the butane diol ester of a dimerized unsaturated fatty acid; oxazoline compounds; and alkoxylated alkyl-substituted mono-amines, diamines and alkyl ether amines, for example, ethoxylated tallow amine and ethoxylated tallow ether amine.

Other known friction modifiers comprise oil-soluble organo-molybdenum compounds. Such organo-molybdenum friction modifiers also provide antioxidant and antiwear credits to a lubricating oil composition. Examples of such oil soluble organo-molybdenum compounds include dithiocarbamates, dithiophosphates, dithiophosphinates, xanthates, thioxanthates, sulfides, and the like, and mixtures thereof Particularly preferred are molybdenum dithiocarbamates, dialkyldithiophosphates, alkyl xanthates, and alkylthioxanthates.

Additionally, the molybdenum compound can be an acidic molybdenum compound. These compounds will react with a basic nitrogen compound as measured by ASTM test D-664 or D-2896 titration procedure and are typically hexavalent Included are molybdic acid, ammonium molybdate, sodium molybdate, potassium molybdate, and other alkaline metal molybdates and other molybdenum salts, e.g., hydrogen sodium molybdate, MoOCl₄, MoO₂Br₂, Mo₂O₃Cl₆, molybdenum trioxide or similar acidic molybdenum compounds.

Among the molybdenum compounds useful in the compositions of this invention are organo-molybdenum compounds of the formula: Mo(ROCS₂)₄ and Mo(RSCS₂)₄, wherein R is an organo group selected from the group consisting of alkyl, aryl, aralkyl, and alkoxyalkyl, generally of from 1 to 30 carbon atoms, preferably 2 to 12 carbon atoms, and most preferably alkyl of 2 to 12 carbon atoms. Especially preferred are the dialkyldithiocarbamates of molybdenum.

Another group of organo-molybdenum compounds useful in the lubricating compositions of this invention are trinuclear molybdenum compounds, especially those of the formula Mo3SₖLₙQ_{z} and mixtures thereof wherein the L are independently selected ligands having organo groups with a sufficient number of carbon atoms to render the compound soluble or dispersible in the oil, n is from 1 to 4, k varies from 4 through 7, Q is selected from the group of neutral electron donating compounds such as water, amines, alcohols, phosphines, and ethers, and z ranges from 0 to 5 and includes non-stoichiometric values. At least 21 total carbon atoms should be present among all the ligand organo groups, such as at least 25, at least 30, or at least 35 carbon atoms.

Pour point depressants, otherwise known as lube oil flow improvers (LOFI), lower the minimum temperature at which the fluid will flow or can be poured. Such additives are well known. Typical of those additives that improve the low temperature fluidity of the fluid are C₈ to C₁₈ dialkyl fumarate/vinyl acetate copolymers, and polymethacrylates. Foam control can be provided by an antifoamant of the polysiloxane type, for example, silicone oil or polydimethyl siloxane.

Some of the above-mentioned additives can provide a multiplicity of effects; thus, for example, a single additive can act as a dispersant-oxidation inhibitor. This approach is well known and need not be further elaborated herein.

In the present invention it may be necessary to include an additive that maintains the stability of the viscosity of the blend. Thus, although polar group-containing additives achieve a suitably low viscosity in the pre-blending stage, it has been observed that some compositions increase in viscosity when stored for prolonged periods. Additives which are effective in controlling this viscosity increase include the long chain hydrocarbons functionalized by reaction with mono- or dicarboxylic acids or anhydrides which are used in the preparation of the ashless dispersants as hereinbefore disclosed.

When lubricating compositions contain one or more of the above-mentioned additives, each additive is typically blended into the base oil in an amount that enables the additive to provide its desired function. Representative effect amounts of such additives, when used in crankcase lubricants, are listed below. All the values listed are stated as weight percent active ingredient.

TABLE 2

| | Weight % | Weight % |
|---|---|---|
| ADDITIVE | (Desirable) | (Preferred) |
| Metal Detergents | 0.1-15 | 0.2-9 |
| Corrosion Inhibitor | 0-5 | 0-1.5 |
| Metal Dihydrocarbyl Dithiophosphate | 0.1-6 | 0.1-4 |
| Antioxidant | 0-5 | 0.01-3 |
| Pour Point Depressant | 0.01-5 | 0.01-1.5 |
| Antifoaming Agent | 0-5 | 0.001-0.15 |
| Supplemental Antiwear Agents | 0-1.0 | 0-0.5 |
| Friction Modifier | 0-5 | 0-1.5 |
| Viscosity Modifier | 0.01-10 | 0.25-3 |
| Basestock | Balance | Balance |

Fully formulated passenger car diesel engine lubricating oil (PCDO) compositions of the present invention preferably have a sulfur content of less than about 0.4 weight %, such as less than about 0.35 weight %, more preferably less than about 0.03 weight %, such as less than about 0.15 weight %. Preferably, the Noack volatility of the fully formulated PCDO (oil of lubricating viscosity plus all additives) will be no greater than 13, such as no greater than 12, preferably no greater than 10. Fully formulated PCDOs of the present invention preferably have no greater than 1200 ppm of phosphorus, such as no greater than 1000 ppm of phosphorus, or no greater than 800 ppm of phosphorus. Fully formulated PCDOs of the present invention preferably have a sulfated ash (SASH) content of about 1.0 weight % or less.

Fully formulated heavy duty diesel engine (HDD) lubricating oil compositions of the present invention preferably have a sulfur content of less than about 1.0 weight %, such as less than about 0.6 weight %, more preferably less than about 0.4 weight %, such as less than about 0.15 weight %. Preferably, the Noack volatility of the fully formulated HDD lubricating oil composition (oil of lubricating viscosity plus all additives) will be no greater than 20, such as no greater than 15, preferably no greater than 12. Fully formulated HDD lubricating oil compositions of the present invention preferably have no greater than 1600 ppm of phosphorus, such as no greater than l 400 ppm of phosphorus, or no greater than 1200 ppm of phosphorus. Fully formulated HDD lubricating oil compositions of the present invention preferably have a sulfated ash (SASH) content of about 1.0 weight % or less.

It may be desirable, although not essential, to prepare one or more additive concentrates comprising additives (concentrates sometimes being referred to as additive packages) whereby several additives can be added simultaneously to the oil to form the lubricating oil composition. A concentrate for the preparation of a lubricating oil composition of the present invention can, for example, contain from about 0.1 to about 16 weight % of phenylenediamine; about 10 to about 40 weight % of a nitrogen-containing dispersant; about 2 to about 20 weight % of an aminic antioxidant and/or a phenolic antioxidant, a molybdenum compound, or a mixture thereof; about 5 to 40 weight % of a detergent; and from about 2 to about 20 weight % of a metal dihydrocarbyl dithiophosphate.

The final composition can employ from 5 to 25 weight %, preferably 5 to 18 weight %, typically 10 to 15 weight %, of the concentrate, the remainder being oil of lubricating viscosity and viscosity modifier.

All weight percents expressed herein (unless otherwise indicated) are based on active ingredient (A.I.) content of the additive, and/or upon the total weight of any additive-package, or formulation which will be the sum of the A.I. weight of each additive plus the weight of total oil or diluent.

### EXAMPLES

This invention will be further understood by reference to the following examples. As noted in Table 3, most of the experimental compounds were prepared by one of two basic methods (A and B). The remaining examples were prepared by minor variants or combinations of these methods, as set forth below. Products were characterized by 300 MHz ¹H NMR, infrared spectroscopy, gas chromatography. Some products were further characterized by GC/MS and ¹³C NMR.

### Examples 1 through 37 and Comparative Examples A through I

The invention is disclosed in Examples 1 through 37. Comparative Examples A through I are also provided.

The compounds of the examples are produced by one of three methods. Method A is exemplified by the details provided below for Examples 6, 9, 15, 27, 29, and 32. Method B is exemplified by Example 13, and Method C by Example 12.

### Method A

The basic method using reductive alkylation of the phenylenediamine with an aldehyde or ketone and sodium triacetoxyborohydride is a modification of the method of Abdel-Majid, A. F. et al., 61 J. ORG. CHEM. 3849-62 (1996). Anyone skilled in the art will recognize that adjustments in stoichiometry, reaction time, and reaction temperature may be required to achieve the desired reaction with varying starting materials. The following examples are illustrative of this “Method A.”

### N,N′-dicyclohexyl-meta-phenylenediamine

Method A was used to produce N,N′-dicyclohexyl-meta-phenylenediamine. This compound has the characteristics reported below for Example 6.

A 250 mL 3-neck flask was fitted with an overhead stirrer, a thermocouple, and a nitrogen inlet. The flask was charged with 2.63 g meta-phenylenediamine, 131 mL tetrahydrofuran, 65 mL xylenes, 12.6 mL cyclohexanone, and 1.4 mL glacial acetic acid. Sodium triacetoxyborohydride, 26 g, was added over 3 h at 35° C. The reaction was taken up in xylenes, extracted with aqueous sodium hydroxide, and washed with water. Solvent was removed by rotary evaporation. The resulting brown oil was taken up in hexanes and cooled to −10° C. Minor solids were removed by vacuum filtration. The liquor was further concentrated, and the product crystals were recovered by filtration mp 84-86° C.

Mixed N,N′-dicyclohexyl-diethyl-meta-toluenediamines Method A was used to produce mixed N,N′-dicyclohexyl-diethyl-meta-toluenediamines. This compound has the characteristics reported below for Example 9.

A 500 mL 3-neck flask was fitted with an overhead stirrer, a thermocouple, and a nitrogen inlet. The flask was charged with 16.0 g Ethacure® 89 mixed diethyl-meta-toluenediamines (available from Chemtura Corporation), 91 mL tetrahydrofuran, 45 mL xylenes, 27.9 mL cyclohexanone, and 5.13 mL glacial acetic acid. Sodium triacetoxyborohydride, 39.8 g, was added over 1 h at 53° C. The reaction was stirred further at 65° C. for 4 h, then 10.2 g additional sodium triacetoxyborohydride was added, and the reaction was stirred for 1 h. An additional 9.0 mL cyclohexanone was added, and the reaction was stirred for 2 h. An additional 5.5 g sodium triacetoxyborohydride was added, and the reaction was stirred for 2 h. The reaction mass was poured into xylenes and extracted with 25% aqueous sodium hydroxide. Solvent was removed by rotary evaporation to yield 30.4 g pale golden colored oil.

### N,N′-dicyclooctyl-N-ethyl para-phenylenediamine and N,N′-dicyclooctyl-N,N′-diethyl para-phenylenediamine

Method A was used to produce N,N′-dicyclooctyl-N-ethyl para-phenylenediamine. This compound has the characteristics reported below for Example 15.

Method A was used to produce N,N′-dicyclooctyl-N,N′-diethyl para-phenylenediamine. This compound has the characteristics reported below for Example 27.

A 250 mL 3-neck flask was fitted with an overhead stirrer, a thermocouple, and a nitrogen inlet. The flask was charged with 3.08 g para-phenylenediamine, 70 mL tetrahydrofuran, and 11.88 g cyclooctanone. Sodium triacetoxyborohydride, 19.8 g, was added over 75 min, and the reaction was heated to 60-65° C. An additional 3.6 g cyclooctanone and 6.2 g sodium triacetoxyborohydride were added. The reaction was stirred at 60° C. for 2 h, and then 3.2 mL glacial acetic acid and 6.0 g sodium triacetoxyborohydride were added. The reaction was stirred for 3 h. The reaction was taken up in xylenes/ethyl acetate, extracted with aqueous sodium hydroxide, and washed with water. Solvents were removed by rotary evaporation. Cyclooctanone was distilled from the reaction under vacuum at 185° C. The product was taken up in heptanes, filtered, concentrated by rotary evaporation, and stored at −10° C. The liquid was filtered again, removing a minor amount of fine solids, and then concentrated by rotary evaporation to yield a dark oil. The oil separated by fractional column chromatography on basic alumina, using 6% ethyl acetate in hexanes, to yield two pale yellow oils (Examples 15 and 24), which crystallized upon prolonged standing.

### N,N,N′-tricyclohexyl-para-phenylenediamine

Method A was used to produce N,N,N′-tricyclohexyl-para-phenylenediamine. This compound has the characteristics reported below for Example 29.

A 2000 mL bottom-out reaction kettle was fitted with an overhead stirrer, a thermocouple, and a nitrogen inlet. The kettle was charged with 30.3 g para-phenylenediamine, and 1030 mL tetrahydrofuran. A 116 mL portion of cyclohexanone was added, followed by 35 mL glacial acetic acid. Sodium triacetoxyborohydride, 65 g, is added over 90 minutes. The mixture was heated to 40° C., and stirred 1 h. A second 65 g charge of sodium triacetoxyborohydride was added over 60 min. The reaction was heated to 60° C., and stirred for 1 h. A third 65 g charge of sodium triacetoxyborohydride was added over 4 h, and the reaction is stirred for an additional 1 h. A 400 mL charge of 1:1 xylenes/ethyl acetate is added, followed by 150 mL water. The reaction was stirred, allowed to settle, and the aqueous layer is drained off. The reaction was extracted with 20% aqueous sodium hydroxide until pH=11, then washed twice with water. Solvent was removed by rotary evaporation. The product was taken up in 450 mL hexanes, and stored overnight at −10° C. A minor amount of fine precipitate was removed by filtration through diatomaceous earth. Solvent was removed by rotary evaporation to yield 93 g reddish oil.

### N,N,N′-tricyclohexyl-N′-heptyl-para-phenylenediamine

Method A was used to produce N,N,N′-tricyclohexyl-N′-heptyl-para-phenylenediamine. This compound has the characteristics reported below for Example 32.

A 500 mL 3-neck flask was fitted with an overhead stirrer, a thermocouple, and a nitrogen inlet. The flask was charged with 40.1 g portion of the above N,N,N′-tricyclohexyl-para-phenylenediamine, 150 mL tetrahydrofuran, and 17.3 g heptaldehyde. Sodium triacetoxyborohydride, 27.8 g, was added to the flask over 1 h. The reaction was stirred at 40° C. for 30 min. The reaction was added to a separatory funnel containing 1:1 xylene: ethyl acetate, and extracted with water, followed by aqueous sodium hydroxide, and then washed three times with water. Solvent was removed by rotary evaporation. The remaining mixture was stripped at 170° C. and 29″ Hg to yield 51 g dark red oil.

### Method B

Method B is represented by the procedure of Example 13.

### N,N′-dicyclohexyl-N-phenyl-para-phenylenediamine

Method B was used to produce N,N′-dicyclohexyl-N-phenyl-para-phenylenediamine. This compound has the characteristics reported below for Example 13.

A 1 L reactor was charged with 156 g N-phenyl-para-phenylenediamine, 336 g cyclohexanone, sulfided platinum on carbon catalyst, and pressurized with 600 psig hydrogen. The reaction was heated to 190° C. for 23.5 h, after which time the pressure had dropped to 93 atm. The reaction mass was cooled, taken up in toluene, and filtered. The product precipitated to yield fine white crystals, mp 110°-112° C.

### Method C

Method C is represented by the procedure of Example 12.

### N-butyl-N,N′-dicyclohexyl-meta-phenylenediamine

Method C was used to produce N-butyl-N,N′-dicyclohexyl-meta-phenylenediamine. This compound has the characteristics reported below for Example 12.

A 100 mL 3-neck flask was fitted with an overhead stirrer, a thermocouple, and a nitrogen inlet. The flask was charged with 7.24 g N,N′-dicyclohexyl-meta-phenylenediamine, 0.121 g Aliquat® 336 (methyl-trioctyl ammonium chloride), 11.4 mL 1-bromobutane, and 16 g 25% sodium hydroxide. The reaction was stirred at 85% for 48 h, after which an additional 2.4 mL of 1-bromobutane was added, and the reaction was stirred for an additional 24 h. The reaction mixture was taken up in xylenes, and the caustic layer removed. The reaction mass was washed three times with water, and volatiles were removed by rotary evaporation. Soft crystals formed on standing, which were recrystallized from hexanes to yield off-white crystals, mp 48-49° C.

### Method D

Method D is represented by the procedure of Example 5.

### N,N′-dicyclohexyl-ortho-phenylenediamine

Method D was used to produce N,N′-dicyclohexyl-ortho-phenylenediamine. This compound has the characteristics reported below for Example 5.

A 100 mL 3-neck flask was fitted with an overhead stirrer, a thermocouple, and a nitrogen inlet. The flask was charged with 5.06 g ortho-phenylenediamine, 30 mL bromocyclohexane, 20.0 g finely ground potassium carbonate, and 15 mL dimethyl sulfoxide. The mixture was stirred for 5 h at 110-120° C. An additional 7.5 mL bromocyclohexane, and 5.0 g finely ground potassium carbonate were added, and the reaction was stirred for 5 h at 110° C.

The reaction mixture was filtered. The filtrate salts were rinsed with xylenes and ethyl acetate. Solvent was removed by rotary evaporation. Remaining volatile components were removed by vacuum distillation. The remaining mixture was stripped at 110° C. and 29″ Hg to yield 9.3 g dark red oil. The stripped product was further purified by flash chromatography on basic alumina. The solvent system was 50:1 hexanes to ethyl acetate. A 5.1 g portion of the desired oily product was isolated.

### Thermo-Oxidation Engine Oil Simulation Test (TEOST) MHT

The engine oil formulation used in the tests contained the following components that are commercially available. There,is no particular restriction on the type and exact composition of the materials in the context of the present invention. The test formulation is given in Table 3.

TABLE 3
Engine Oil Test Formulation
Composition

Base oil
Overbased detergents
Antiwear
Succinimide dispersant
VI improver
Friction Modifier
Secondary diaryl amine antioxidant
Experimental Deposit/Oxidation Control Additive

The significant reduction in deposits that results from the usage of a cycloalkyl-phenylenediamine, according to the practice of this invention, has been convincingly demonstrated in an engine oil formulation by the Mid-High Temperature Thermo-oxidation Engine Oil Simulation Test (TEOST) MHT. This test is an accelerated oxidation test. The test determines the mass of a deposit formed on a specially constructed steel rod by stressing continuously a repetitive passage of 8.5 of test oil under thermal-oxidative and catalytic conditions. The instrument used was manufactured by Tannas Co. and has a typical repeatability of 0.15(x+16) mg wherein x is the mean of two or more repeated test results. The TEOST test conditions are listed in Table 4. The lower the amount of deposits obtained, the better the oxidation stability of the oil.

TABLE 4
TEOST MHT Test Conditions

| | Settings |
|---|---|
| Test Parameters | Test duration 24 hours |
| Rod Temperature | 285° C. |
| Sample size | 8.5 g (mixture of 8.4 g of oil and 0.1 g of catalyst) |
| Sample flow rate | 0.25 g/min |
| Flow rate (dry air) | 10 mL/min |
| Catalyst | Oil soluble mixture containing Fe, Pb, and Sn |

The secondary diarylamine used in the test was a complex mixture of predominantly mono-, di- and tri-nonyl diphenyl amines currently sold under the trade designation Naugalube® 438L, which is commercially available from Chemtura Corporation. The total amount of added deposit control/antioxidants, including the secondary diarylamine and the substituted phenylenediamine according to the practice of this invention, was 1.25 weight percent in each blend.

Comparative Example A contained only the 0.75% secondary amine base-treat level of antioxidant, with no cycloalkyl phenylenediamine deposit control/antioxidant additive. Comparative Example B contained 1.25% of secondary amine, substituting additional secondary diphenylamine for the phenylenediamine deposit control/antioxidant additive. Comparative Example C contained the 0.75% secondary amine base-treat level of antioxidant, with 0.5% Naugalube® 531 antioxidant substituted for the cycloalkyl phenylenediamine deposit control/antioxidant additive. Naugalube® 531 antioxidant is a mixture of C₇-C₉ esters of 3,5-di-tert-butyl-hydroxycinnamic acid and is commercially available from Chemtura Corporation. Comparative Example D contained the 0.75% secondary amine base-treat level of antioxidant, with 0.5% Flexzone® 4 L antioxidant substituted for the cycloalkyl phenylenediamine deposit control/antioxidant additive. Flexzone® 4 L antioxidant is N,N′-bis(methylpentyl)-para-phenylenediamine and is commercially available from Chemtura Corporation. Comparative Examples E-I were prepared according to Method A above.

### Inventive Examples 1 through 38

| Ex. | Method | | TEOST |
|---|---|---|---|
| No. | of Prep. | | (mg deposit) |
| 1 | B | N-(1-methyldecyl)-N′-(2,2,6,6-tetramethyl-4-piperdinyl)-p-phenylenediamine | 23 |
| 2 | A | N-cyclohexyl-N,N′-di(sec-butyl)-p-phenylenediamine | 31 |
| 3 | A | Mixed N-cyclohexyl-N′-phenyl-o-phenylenediamine and | 39 |
| | | N-cyclohexyl-N-phenyl-o-phenylenediamine | |
| 4 | A | N,N′-dicyclohexyl-p-phenylenediamine | 11 |
| 5 | D | N,N′-dicyclohexyl-o-phenylenediamine | 42 |
| 6 | A | N,N′-dicyclohexyl-m-phenylenediamine | 8 |
| 7 | A | N,N′-dicyclohexyl-m-toluenediamine | 12 |
| 8 | A | N,N′-dicyclooctyl-p-phenylenediamine | 17 |
| 9 | A | dicyclohexyl-diethyl-m-toluenediamines | 27 |
| 10 | A | N,N′-bis(3,3,5-trimethylcyclohexyl)-p-phenylenediamine | 24 |
| 11 | A | N,N′-dicyclohexyl-N-isopropyl-p-phenylenediamine | 9 |
| 12 | C | N,N′-dicyclohexyl-N-butyl-m-phenylenediamine | 31 |
| 13 | B | N,N′-dicyclohexyl-N′-phenyl-p-phenylenediamine | 34 |
| 14 | B | N-cyclohexyl-N-(2-ethylhexyl)-N′-phenyl-p-phenylenediamine | 18 |
| 15 | A | N,N′-dicyclooctyl-N-ethyl-p-phenylenediamine | 17 |
| 16 | A | N,N′-dicyclohexyl-N,N′-diethyl-p-phenylenediamine | 23 |
| 17 | A | N,N′-dicyclohexyl-N,N′-dipropyl-p-phenylenediamine | 24 |
| 18 | A | N,N′-dicyclohexyl-N,N′-dibutyl-m-phenylenediamine | 12 |
| 19 | A | N,N′-dicyclohexyl-N,N′-dibutyl-p-phenylenediamine | 23 |
| 20 | A | N,N′-dicyclohexyl-N,N′-di-iso-butyl-p-phenylenediamine | 24 |
| 21 | A | N,N′-dicyclohexyl-N,N′-di-sec-butyl p-phenylenediamine | 39 |
| 22 | A | N,N′-dicyclohexyl-N-(1,3-dimethylbutyl)-p-phenylenediamine | 26 |
| 23 | A | N,N′-dicyclohexyl-N,N′-diheptyl-p-phenylenediamine | 35 |
| 24 | A | N,N′-dicyclohexyl-N,N′-difurfuryl-p-phenylenediamine | 26 |
| 25 | A | N,N′-dicyclohexyl-N,N′-dibenzyl-p-phenylenediamine | 22 |
| 26 | B | N,N′-dicyclohexyl-N′-phenyl-N′-heptyl-p-phenylenediamine | 44 |
| 27 | A | N,N′-dicyclooctyl-N,N′-diethyl-p-phenylenediamine | 22 |
| 28 | A | N,N,N′-tricyclopentyl-p-phenylenediamine | 7 |
| 29 | A | N,N,N′-tricyclohexyl-p-phenylenediamine | 9 |
| 30 | A | N,N,N′-tricyclohexyl-N′-propyl-p-phenylenediamine | 11 |
| 31 | A | N,N,N′-tricyclohexyl-N′-butyl-p-phenylenediamine | 11 |
| 32 | A | N,N,N′-tricyclohexyl-N′-heptyl-p-phenylenediamine | 30 |
| 33 | A | N-(2-ethylhexyl)-N,N′,N′-tricyclohexyl-p-phenylenediamine | 43 |
| 34 | A | N,N,N′-tricyclohexyl-N′-phenyl-p-phenylenediamine | 44 |
| 35 | A | Mixed cyclopentyl/cyclohexyl tetra-N-substituted-p-phenylenediamines | 29 |
| 36 | A | N,N,N′,N′-tetracyclohexyl-p-phenylenediamine | 32 |
| 37 | A | N,N′-bis(2-decalinyl)-p-phenylenediamine | 15 |
| 38 | B | Mixed N,N′-dicyclohexyl-4-isobutenyl-6-isobutyl-1,3-phenylenediamine | 15 |
| | | and N,N′-dicyclohexyl-4,6-diisobutyl-1,3-phenylenediamine | |

### Comparative Examples A through I

| | Method | | TEOST |
|---|---|---|---|
| Ref. | of Prep. | | (mg deposit) |
| A | Comm.Prod. | Naugalube 438L dinonyl diphenylamine antioxidant at 0.75 wt % total | 73 |
| B | Comm.Prod. | Naugalube 438L dinonyl diphenylamine antioxidant at 1.25 wt % total | 51 |
| C | Comm.Prod. | Naugalube 531 3,5-di-t-butyl-hydroxycinnamic acid, C₇-C₉ branched ester | 55 |
| D | Comm.Prod. | Flexzone 4L N,N′-bis(methylpentyl)-p-phenylenediamine | 62 |
| E | A | N-heptyl-N,N′,N′-triisopropyl-p-phenylenediamine | 55 |
| F | A | N,N,N′-triisopropyl-p-phenylenediamine | 62 |
| G | A | N,N,N′,N′-tetraheptyl-p-phenylenediamine | 53 |
| H | B | N,N′-diisopropyl-p-phenylenediamine | 61 |
| I | A | N,N′-di-sec-butyl-p-phenylenediamine | 58 |

The deposits obtained for Inventive Examples 1 through 37 are significantly lower than the deposits obtained for Comparative Examples A through I. This demonstrates that the lubricating oil compositions containing appropriate mixtures of the deposit control/antioxidant blends of the present invention have superior oxidative stabilities to produce smaller amounts of deposits in the TEOST.

The deposit obtained for Inventive Example 4 (11 mg) is much less than the deposits obtained for the corresponding acyclic Comparative Examples H and I (61, 58 mg). The deposits obtained for Inventive Examples 26 and 27 (9, 30 mg) are also much less than the deposits obtained for the corresponding acyclic Comparative Examples F and E. This result is entirely unexpected, as in each case the substituents on the phenylenediamine nitrogens all have the same secondary carbon substitution. One would not have anticipated that incorporating the sec-alkyl group into an alicyclic ring would have had such a profound effect on deposit control. These results are all the more surprising, as the cyclic compounds are of slightly higher molecular weight, and therefore the additive formulations contain fewer mol equivalents of the active deposit control agents.

In view of the many changes and modifications that can be made without departing from principles underlying the present invention, reference should be made to the appended claims for an understanding of the scope of the protection to be afforded the invention.

The disclosures of all patents, articles and other materials described herein are hereby incorporated, in their entirety, into this specification by reference. Compositions described as “comprising” a plurality of defined components are to be construed as including compositions formed by admixing the defined plurality of defined components. The principles, preferred embodiments, and modes of operation of the present invention have been described in the foregoing specification. What the Applicants submit is their invention, however, is not to be construed as limited to the particular embodiments disclosed, since the disclosed embodiments are regarded as illustrative rather than limiting. Changes can be made by those skilled in the art without departing from the spirit of the invention.

What is claimed is:

1. A composition comprising a fluid subject to oxidative degradation and an amount of at least one N-cycloalkyl-substituted phenylenediamine additive sufficient to stabilize said fluid against the formation of deposits, wherein said additive is of the structure: 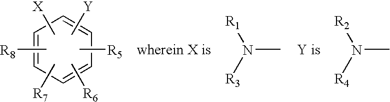
R₁ is a cycloalkyl ring selected from the group consisting of: 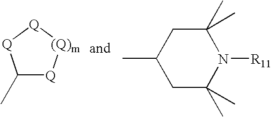
wherein
R₅, R₆, R₇, and R₈ are independently selected from the group consisting of hydrogen, linear and branched alkyl groups of from 1 to 20 carbon atoms, cycloalkyl, allyl, methyallyl, isobutenyl, alkenyl, furfuryl, benzyl, alkaryl, and phenyl;
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
and
m is an integer of 1 to 8; and
wherein, when X and Y are para to each other, R₂, R₃, and R₄ are in an interrelated relationship selected from the group consisting of:
(A) R₂ is a cycloalkyl ring independently selected from the group consisting of: 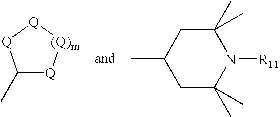
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
R₃ is an independently selected cycloalkyl ring of the structure: 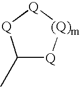
wherein Q and m are as defined above;
and
R₄ is selected from the group consisting of linear or branched alkyl of from 1 to about 36 carbon atoms, aryl, alkaryl, cycloalkyl, and alkenyl of from 3 to about 36 carbon atoms, all optionally containing a nitrogen, oxygen, or sulfur atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not —CH₂CH₂— or —CH₂CH(CH₃)—;
(B) R₂ is a cycloalkyl ring independently selected from the group consisting of: 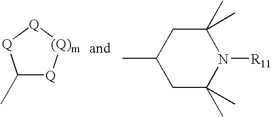
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and
wherein Q and m are as defined above;
and
R₃ and R₄ are independently selected from the group consisting of linear or branched alkyl of from 1 to about 36 carbon atoms, aryl, alkaryl, cycloalkyl, and alkenyl of from 3 to about 36 carbon atoms, all optionally containing a nitrogen atom, hydrogen, and ZCO₂_{R}₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, that Z is not —CH₂CH₂— or —CH₂CH(CH₃)—, and that R₃ and R₄ are not both hydrogen;
(C) R₃ is a cycloalkyl ring independently selected from the group consisting of: 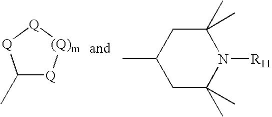
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
and
R₂ and R₄ are independently selected from the group consisting of linear or branched alkyl of from 1 to about 36 carbon atoms, aryl, alkaryl, cycloalkyl, and alkenyl of from 3 to about 36 carbon atoms, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, and that Z is not —CH₂CH₂— or —CH₂CH(CH₃)—;
(D) R₂, R₃, and R₄ are independently selected from the group consisting of alkyls of from 3 to about 36 carbon atoms, alkenyls of from 3 to about 36 carbon atoms, aryl, alkaryl, and cycloalkyl, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, that Z is not —CH₂CH₂— or —CH₂CH(CH₃)—; and,
(1) at least two of R₂, R₃, and R₄ are independently selected from the group consisting of alkyls of from 3 to about 36 carbon atoms, alkenyls of from 3 to about 36 carbon atoms, wherein said alkyls or alkenyls are branched on the carbon bonded to the phenylenediamine nitrogen; and that
(2) when R₁ is a 2,2,6,6-tetramethylpiperidinyl, R₃ can be hydrogen; and
wherein, when X and Y are ortho or meta to each other, R₂, R₃, and R₄ are in an interrelated relationship selected from the group consisting of:
(E) R₂ is a cycloalkyl ring independently selected from the group consisting of: 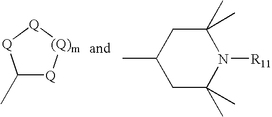
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
and
R₃ and R₄ are independently selected from the group consisting of linear or branched alkyl of from 1 to about 36 carbon atoms, aryl, alkaryl, and cycloalkyl, all optionally containing a nitrogen, oxygen, or sulfur atom, hydrogen, alkenyl of from 3 to about 36 carbon atoms, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not —CH₂CH₂— or —CH₂CH(CH₃)—, and that R₃ and R₄ are not both hydrogen;
(F) R₂, R₃, and R₄ are independently selected from the group consisting of linear or branched alkyl of from 1 to about 36 carbon atoms and cycloalkyl, all optionally containing a nitrogen, oxygen, or sulfur atom, hydrogen, aryl, alkaryl, alkenyl of from 3 to about 36 carbon atoms, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S;provided that Z is not —CH₂CH₂— or —CH₂CH(CH₃)—; and wherein said phenylenediamine is in the form of a free base, or an oil-soluble salt.

2. The composition of claim 1 wherein the N-cycloalkyl substituted phenylenediamine has the structure: 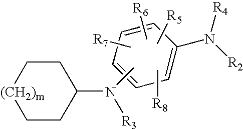
where m is 0, 1, 2, or 3, R₂, R₃ and R₄ are independently selected from the group consisting of alkyl, branched alkyl, cycloalkyl, alkenyl of 3 to about 36 carbons, alkaryl, and aryl, all optionally containing an N, O, or S atom, and hydrogen; and R₅, R₆, R₇, and R₈ are independently selected from the group consisting of linear or branched alkyl of 1 to about 20 carbons and hydrogen; with the proviso that at least two of R₂, R₃, and R₄ are independently alkyls branched on the carbon bonded to the phenylenediamine nitrogen.

3. The composition of claim 1 wherein the N,N′-dicycloalkyl substituted phenylenediamine has the structure: 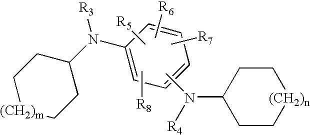
where m and n are independently 0, 1, 2, or 3, R₃ and R₄ are independently selected from the group consisting of alkyl, branched alkyl, cycloalkyl, alkenyl of 3 to about 36 carbons, alkaryl, and aryl; optionally containing an N, O, or S atom, and hydrogen; and R₅, R₆, R₇, and R₈ are independently selected from the group consisting of linear or branched alkyls of 1 to about 20 carbons, and hydrogen.

4. The composition of claim 1 wherein the N-cycloalkyl-substituted phenylenediamine has the structure: 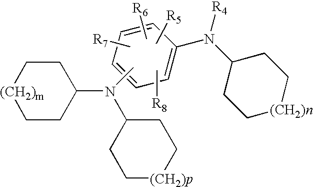
where m, n, and p are independently 0, 1, 2, or 3, and R₄ is selected from the group consisting of linear or branched alkyl, cycloalkyl, allyl, alkenyl, alkaryl, and aryl, all optionally containing a N, O, or S atom; and R₅, R₆, R₇, and R₈ are independently selected from the group consisting of linear or branched alkyl of 1 to about 20 carbons, and hydrogen.

5. The composition of claim 1 further comprising an organic material subject to oxidative, thermal, or light-induced degradation.

6. The composition of claim 5 wherein the organic material is selected from the group consisting of natural functional fluids, synthetic functional fluids, and mixtures thereof.

7. The composition of claim 6 wherein the functional fluid is selected from the group consisting of lubricating oil, grease, fuel, natural oils, and mixtures thereof.

8. The composition of claim 7 wherein the functional fluid is a lubricating oil selected from the group consisting of engine oils, transmission fluids, hydraulic fluids, gear oils, marine cylinder oils, compressor oils, refrigeration lubricants, and mixtures thereof.

9. The composition of claim 8 wherein the lubricating oil has a viscosity of about 1.5 to about 2000 centistokes (cSt) at 100° C.

10. The composition of claim 8 further comprising at least one lubricating oil additive.

11. The composition of claim 10 wherein the lubricating oil additive is selected from the group consisting of antioxidants, anti-wear agents, detergents, rust inhibitors, dehazing agents, demulsifying agents, metal deactivating agents, friction modifiers, pour point depressants, antifoaming agents, co-solvents, package compatibilizers, corrosion-inhibitors, ashless dispersants, dyes, extreme pressure agents, and mixtures thereof.

12. The composition of claim 10 wherein the lubricating oil additive is selected from the group consisting of alkylated diphenylamines, alkylated hindered phenolics, alkylated substituted or unsubstituted phenylenediamines, alkylated oil soluble copper compounds, alkylated sulfur-containing compounds known to impart oxidation stability, and mixtures thereof.

13. The composition of claim 12 wherein the alkylated sulfur-containing compound is selected from the group consisting of a phenothiazine, sulfurized olefin, thiocarbamate, sulfur-bearing hindered phenolic, zinc dialkyldithiophosphate, and mixtures thereof.

14. The composition of claim 10 wherein the lubricating oil additive is selected from the group consisting of fatty acid esters or amides, organo molybdenum compounds, molybdenum dialkyldithiocarbamates, molybdenum dialkyl dithiophosphates, molybdenum disulfides, tri-molybdenum cluster dialkyldithiocarbamates, non-sulfur molybdenum compounds, and mixtures thereof.

15. The composition of claim 10 wherein the lubricating oil additive is selected from the group consisting of zinc dialkyldithiophosphates, zinc diaryldithiophosphates, dialkyldithiophosphate esters, diaryl dithiophosphate esters, and mixtures thereof.

16. The composition of claim 10 wherein the lubricating oil additive has a phosphorus content of less than about 0.08 weight percent.

17. The composition of claim 10 further comprising at least one diaromatic amine compound in an amount of about 0.1 to about 10 weight %, based on the total weight of the composition.

18. The composition of claim 5 comprising a mixture of at least two N-cycloalkyl substituted phenylenediamine compounds.

19. The composition of claim 6 further comprising an additive package comprising about 1 to about 75 weight% of said N-cycloalkyl substituted phenylenediamine compound.

20. A method for stabilizing an organic material subject to oxidative, thermal, or light-induced degradation comprising adding to the organic material a stabilizing amount of at least one N-cycloalkyl-substituted phenylenediamine additive of the structure: 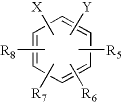
wherein
X is 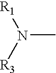
Y is 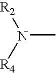
R₁ is a cycloalkyl ring selected from the group consisting of: 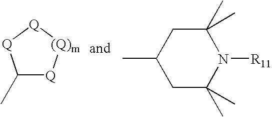
wherein
R₅, R₆, R₇, and R₈ are independently selected from the group consisting of hydrogen, linear and branched alkyl groups of from 1 to 20 carbon atoms, cycloalkyl, allyl, methyallyl, isobutenyl, alkenyl, furfuryl, benzyl, alkaryl, and phenyl;
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
and
m is an integer of 1 to 8; and
wherein, when X and Y are para to each other, R₂, R₃, and R₄ are in an interrelated relationship selected from the group consisting of:
(A) R₂ is a cycloalkyl ring independently selected from the group consisting of: 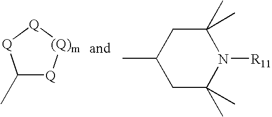
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
R₃ is an independently selected cycloalkyl ring of the structure: 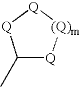
wherein Q and m are as defined above;
and
R₄ is selected from the group consisting of linear or branched alkyl of from 1 to about 36 carbon atoms, aryl, alkaryl, cycloalkyl, and alkenyl of from 3 to about 36 carbon aims, all optionally containing a nitrogen, oxygen, or sulfur atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not—CH₂CH₂— or —CH₂CH(CH₃)—;
(B) R₂ is a cycloalkyl ring independently selected from the group consisting of: 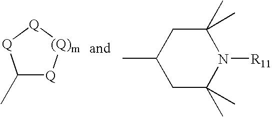
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and
wherein Q and m are as defined above;
and
R₃ and R₄ are independently selected from the group consisting of linear or branched alkyl of from 1 to about 36 carbon atoms, aryl, alkaryl, cycloalkyl and alkenyl of from 3 to about 36 carbon atoms, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, that Z is not —CH₂CH₂— or —CH₂CH(CH₃)—, and that R₃ and R₄ are not both hydrogen;
(C) R₃ is a cycloalkyl ring independently selected from the group consisting of: 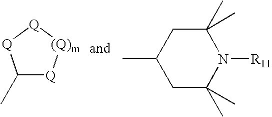
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
and
R₂ and R₄ are independently selected from the group consisting of linear or branched alkyl of from 1 to about 36 carbon atoms, aryl, alkaryl, cycloalkyl, and alkenyl of from 3 to about 36 carbon atoms, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, and that Z is not —CH₂CH₂— or —CH₂CH(CH₃)—;
(D) R₂, R₃, and R₄ are independently selected from the group consisting of alkyls of from 3 to about 36 carbon atoms, alkenyls of from 3 to about 36 carbon atoms, aryl, alkaryl, and cycloalkyl, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, that Z is not —CH₂CH₂— or —CH₂CH(CH₃)—; and
(1) at least two of R₂, R₃, and R₄ are independently selected from the group consisting of alkyls of from 3 to about 36 carbon atoms, alkenyls of from 3 to about 36 carbon atoms, wherein said alkyls or alkenyls are branched on the carbon bonded to the phenylenediamine nitrogen; and that
(2) when R₁ is a 2,2,6,6-tetramethylpiperidinyl, R₃ can be hydrogen; and
wherein, when X and Y are ortho or meta to each other, R₂, R₃, and R₄ are in an interrelated relationship selected from the group consisting of:
(E) R₂ is a cycloalkyl ring independently selected from the group consisting of: 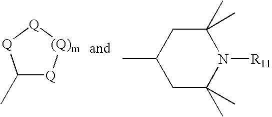
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
and
R₃ and R₄ are independently selected from the group consisting of linear or branched alkyl of from 1 to about 36 carbon atoms, aryl, alkaryl, and cycloalkyl, all optionally containing a nitrogen, oxygen, or sulfur atom, hydrogen, alkenyl of from 3 to about 36 carbon atoms, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not—CH₂CH₂— or —CH₂CH(CH₃)—, and that R₃ and R₄ are not both hydrogen;
(F) R₂, R₃, and R₄ are independently selected from the group consisting of linear or branched alkyl of from 1 to about 36 carbon atoms and cycloalkyl, all optionally containing a nitrogen, oxygen, or sulfur atom, hydrogen, aryl, alkaryl, alkenyl of from 3 to about 36 carbon atoms, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S;provided that Z is not —CH₂CH₂— or —CH₂CH(CH₃)—;
and wherein said phenylenediamine is in the form of a free base, or an oil-soluble salt.

21. A compound comprising the structure: 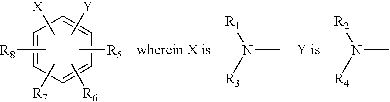
R₁ is a cycloalkyl ring selected from the group consisting of: 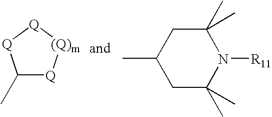
wherein
R₅, R₆, R₇, and R₈ are independently selected from the group consisting of hydrogen, linear and branched alkyl groups of from 1 to 20 carbon atoms, cycloalkyl, allyl, methyallyl, isobutenyl, alkenyl, furfuryl, benzyl, alkaryl, and phenyl;
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
and
m is an integer of 1 to 8; and
wherein, when X and Y are para to each other, R₂, R₃, and R₄ are in an interrelated relationship selected from the group consisting of:
(A) R₂ is a cycloalkyl ring independently selected from the group consisting of: 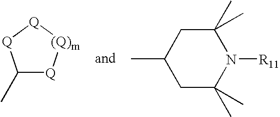
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
R₃ is an independently selected cycloalkyl ring of the structure: 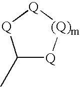
wherein Q and m are as defined above;
and
R₄ is selected from the group consisting of linear or branched alkyl of from 1 to about 36 carbon atoms, aryl, alkaryl, cycloalkyl, and alkenyl of from 3 to about 36 carbon atoms, all optionally containing a nitrogen, oxygen, or sulfur atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not —CH₂CH₂— or —CH₂CH(CH₃)—;
(B) R₂ is a cycloalkyl ring independently selected from the group consisting of: 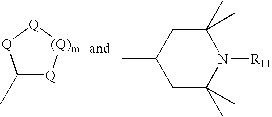
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and
wherein Q and m are as defined above;
and
R₃ and R₄ are independently selected from the group consisting of linear or branched alkyl of from 1 to about 36 carbon atoms, aryl, alkaryl, cycloalkyl, and alkenyl of from 3 to about 36 carbon atoms, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, that Z is not —CH₂CH₂— or —CH₂CH(CH₃)—, and that R₃ and R₄ are not both hydrogen;
(C) R₃ is a cycloalkyl ring independently selected from the group consisting of: 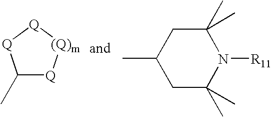
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
and
R₂ and R₄ are independently selected from the group consisting of linear or branched alkyl of from 1 to about 36 carbon atoms, aryl, alkaryl, cycloalkyl, and alkenyl of from 3 to about 36 carbon atoms, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, and that Z is not —CH₂CH₂— or —CH₂CH(CH₃)—;
(D) R₂, R₃, and R₄ are independently selected from the group consisting of alkyls of from 3 to about 36 carbon atoms, alkenyls of from 3 to about 36 carbon atoms, aryl, alkaryl, and cycloalkyl, all optionally containing a nitrogen atom, hydrogen, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that R₃ and R₄ are not 2-hydroxyethyl, that Z is not —CH₂CH₂— or —CH₂CH(CH₃)—; and
(1) at least two of R₂, R₃, and R₄ are independently selected from the group consisting of alkyls of from 3 to about 36 carbon atoms, alkenyls of from 3 to about 36 carbon atoms, wherein said alkyls or alkenyls are branched on the carbon bonded to the phenylenediamine nitrogen; and that
(2) when R₁ is a 2,2,6,6-tetramethylpiperidinyl, R₃ can be hydrogen; and
wherein, when X and Y are ortho or meta to each other, R₂, R₃, and R₄ are in an interrelated relationship selected from the group consisting of:
(E) R₂ is a cycloalkyl ring independently selected from the group consisting of: 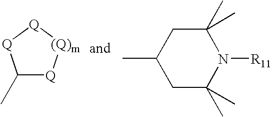
wherein
each Q is C(R₉)(R₁₀), wherein each R₉ and R₁₀ is independently selected from the group consisting of hydrogen, linear and branched alkyl groups of from 1 to 20 carbon atoms, and cycloalkyl;
wherein R₁₁ is H, linear and branched alkyl groups of from 1 to 20 carbon atoms, cycloalkyl, aryl, alkaryl, and O; and wherein Q and m are as defined above;
and
R₃ and R₄ are independently selected from the group consisting of linear or branched alkyl of from 1 to about 36 carbon atoms, aryl, alkaryl, and cycloalkyl, all optionally containing a nitrogen, oxygen, or sulfur atom, hydrogen, alkenyl of from 3 to about 36 carbon atoms, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S; provided that Z is not—CH₂CH₂— or —CH₂CH(CH₃)—, and that R₃ and R₄ are not both hydrogen;
(F) R₂, R₃, and R₄ are independently selected from the group consisting of linear or branched alkyl of from 1 to about 36 carbon atoms and cycloalkyl, all optionally containing a nitrogen, oxygen, or sulfur atom, hydrogen, aryl, alkaryl, alkenyl of from 3 to about 36 carbon atoms, and ZCO₂R₁₁, wherein Z is an alkylene or alkenyl group of from 1 to about 36 carbons, and R₁₁ is selected from the group consisting of linear or branched alkyl, cycloalkyl, aryl, and alkaryl, all optionally containing O, N, or S;provided that Z is not —CH₂CH₂— or —CH₂CH(CH₃)—;
and wherein said phenylenediamine is in the form of a free base, or an oil-soluble salt.

22. A compound selected from the group consisting of:
N-(1-methyldecyl)-N′-(2,2,6,6-tetramethyl-4-piperdinyl)-p-phenylenediamine;
N-cyclohexyl-N-phenyl-o-phenylenediamine;
N,N′-dicyclohexyl-m-phenylenediamine;
N,N′-dicyclohexyl-m-toluenediamine;
N,N′-dicyclohexyl-3,5-diethyl-2,4-toluenediamine;
N,N′-dicyclohexyl-3,5-diethyl-2,6-toluenediamine;
N-cyclohexyl-N-(2-ethylhexyl)N′-phenyl-p-phenylenediamine;
N-cyclohexyl-N,N′-di(sec-butyl)-p-phenylenediamine;
N,N′-dicyclooctyl-N-ethyl-p-phenylenediamine;
N,N′-dicyclohexyl-N-isopropyl-p-phenylenediamine;
N,N′-dicyclohexyl-N-isobutyl-p-phenylenediamine;
N,N′-dicyclohexyl-N-butyl-m-phenylenediamine;
N,N′-dicyclohexyl-N-butyl-p-phenylenediamine;
N,N′-dicyclohexyl-N-isobutyl-m-phenylenediamine;
N,N′-dicyclohexyl-N-isobutyl-m-phenylenediamine;
N,N′-dicyclohexyl-N-heptyl-m-phenylenediamine;
N,N′-dicyclohexyl-N-heptyl-p-phenylenediamine;
N,N′-dicyclohexyl-N-phenyl-p-phenylenediamine;
N,N-dicyclohexyl-N′-phenyl-p-phenylenediamine;
N,N′-dicyclohexyl-N,N′-difurfuryl-p-phenylenediamine;
N,N′-dicyclohexyl-N,N′-dibenzyl-p-phenylenediamine;
N,N′-dicyclohexyl-N′-phenyl-N-heptyl-p-phenylenediamine;
N,N′-dicyclohexyl-N,N′-diethyl-p-phenylenediamine;
N,N′-dicyclohexyl-N,N′-dibutyl-m-phenylenediamine;
N,N′-dicyclohexyl-N,N′-dibutyl-p-phenylenediamine;
N,N′-dicyclohexyl-N,N′-diisobutyl-m-phenylenediamine;
N,N′-dicyclohexyl-N,N′-diisobutyl-p-phenylenediamine;
N,N′-dicyclohexyl-N,N′-diheptyl-m-phenylenediamine;
N,N′-dicyclohexyl-N,N′-diheptyl-p-phenylenediamine;
N,N′-dicyclooctyl-N,N′-diethyl-p-phenylenediamine;
N,N,N′-tricyclopentyl-p-phenylenediamine;
N,N,N′-tricyclohexyl-p-phenylenediamine;
N,N,N′-tricyclohexyl-N′-heptyl-p-phenylenediamine;
N,N,N′-tricyclohexyl-N′-propyl-p-phenylenediamine;
N,N,N′-tricyclohexyl-N′-butyl-p-phenylenediamine;
N-(2-ethylhexyl)-N,N′,N′-tricyclohexyl-p-phenylenediamine;
N,N,N′-tricyclohexyl-N′-phenyl-p-phenylenediamine;
N,N,N′,N′-tetracyclohexyl-p-phenylenediamine;
N,N′-bis(2-decalinyl)-p-phenylenediamine;
N,N′-dicyclohexyl-4,6-diisobutyl-1,3-phenylenediamine; and
N,N′-dicyclohexyl-4-isobutenyl-6-isobutyl-1,3-phenylenediamine.
